(19) United States
(12) Patent Application Publication
(10) Pub. No.: US 2024/0341590 A1
(43) Pub. Date: Oct. 17, 2024

(54) METHODS AND SYSTEMS FOR ALIGNING AN EYE FOR MAKING A DETERMINATION ABOUT THE EYE USING REMOTE OR TELEHEALTH PROCEDURES

(71) Applicant: Ohio State Innovation Foundation, Columbus, OH (US)

(72) Inventor: Melissa Bailey, Westerville, OH (US)

(21) Appl. No.: 18/635,747

(22) Filed: Apr. 15, 2024

Related U.S. Application Data
(60) Provisional application No. 63/459,116, filed on Apr. 13, 2023.

(51) Int. Cl. A61B 3/103; A61B 3/15

(52) U.S. Cl. CPC A61B 3/103; A61B 3/152

(57) ABSTRACT

Disclosed herein are methods and systems for making a determination about an eye. The eye is aligned with an image acquisition device using a software application executing on the image acquisition device that provides visual and/or audible indicators through peripherals of the image acquisition device. The acquired image(s) is then transmitted to a cloud-based network where a determination about the eye is made based on the image(s). In some instances, a trained person has an ability to access, review, confirm or modify the determination made about the eye.

### CROSS-REFERENCE TO RELATED APPLICATIONS

This application claims the benefit of U.S. provisional patent application No. 63/459,116, filed on Apr. 13, 2023, and titled “METHODS AND SYSTEMS FOR ALIGNING AN EYE FOR MAKING A DETERMINATION ABOUT THE EYE USING REMOTE OR TELEHEALTH PROCEDURES,” the disclosure of which is expressly incorporated herein by reference in its entirety.

### BACKGROUND

There are many existing devices that are used to detect the optical quality of the eye or other optical systems, including: autorefractors/ophthalmic refractometers, aberrometers, etc. All of the existing devices work by using a light source to illuminate the eye. Many devices, including the vast majority of autorefractors, use an infrared light source, but visible light sources are also used. Anyone who has used a standard camera with a flash will know that light from the flash will reflect off of the retina during photography. This reflected light will make the pupil appear red in a photograph of a human eye or appear greenish in a photograph of many animals' eyes. The reflected light will also have a particular pattern that is dependent upon the eye's optical distortions. Many existing/previous autorefractors or aberrometers are based on this principle, i.e., shining a light into the eye and then detecting the pattern of the reflected light after it has been distorted by the eye. The devices vary in the configuration or type of light source or in how the reflected light is detected (single images, lenslet arrays, telescope combined with a lenslet array, etc.). However, in each of those cases, a light is shined into the eye and then the magnitude of the refractive error is determined, and this is often based on the intensity slope of the light (brighter at either the top or the bottom of the pupil) that is reflected off of the retina and back out of the eye.

Clinical measurements of tropia and phoria are used across multiple healthcare fields to detect vision problems, either naturally-occurring or due to traumatic brain injury, that would lead to double vision. The predominant, current method for measuring eye alignment, called the cover test, is manual, technically-difficult, and tedious. Other widely-used clinical methods that are automated only determine whether tropia is present, but these methods do not detect the more common deviation in alignment, phoria.

All current methods of measuring eye alignment, either manual or automated, also lack the ability to detect whether or not the subject is accommodating, or focusing the eyes as if to look at an object closer than optical infinity. It would be important for the individual measuring eye alignment to know whether or not someone is accommodating because over- or under-accommodating during a tropia or phoria measurement, will affect the lateral position of the eye, i.e., how much the eyes are turned inwards or outwards.

Additionally, measuring the thickness of the retinal nerve fiber layer, an important health measurement of the eye, must currently be assessed clinically with an expensive, not-portable instrumentation. This makes the testing inaccessible to patients in a vision screening environment, or to those who are in a remote area where there are no health care providers available to make the measurement. A smart device could measure ambient room lighting (in the visible range, i.e., not ultraviolet, infrared, etc.) that is reflected back from the retina (i.e., red eye reflex). According to the literature, visible lighting is likely reflected from the first layer of the retina, the retinal nerve fiber layer. The light returning from this layer is known to be reflected in manner that is polarized. Other existing devices already measure the health of this layer of the retina by measuring the amount of polarized light that is reflected from this layer, and especially by looking at particular regions. A simple polarizing filter in front of the camera lens and take two pictures, one with the filter oriented in one direction and one with the filter oriented in a second orientation. The two images would be compared to measure the retinal nerve fiber layer health, based on the image brightness comparison. It is also likely that the person's overall age would be used as a calibrating factor.

Furthermore, in many instances, in order to be examined using devices such as: autorefractors/ophthalmic refractometers, aberrometers, etc., a patient must travel to an office where a trained person (i.e., a licensed optometrist, ophthalmologist, etc.) performs the examination using the device, which can be challenging to achieve in areas where there are few trained professionals, or in the case of a vision screening that will be performed by a layperson.

Therefore, methods, apparatus and systems are desired that improve the detection of an optical quality of the eye or other optical system and that overcome challenges in the art, some of which are described above. In particular, systems and methods are desired that allow detection of an optical quality of the eye or other optical system remotely using telehealth procedures or when used by a layperson in a vision screening.

### SUMMARY

Described herein are devices and methods to measure optical distortions in the eye by monitoring the intensity of a first color of light versus intensity of a second color of light within the pupil of a subject under ambient lighting conditions, which is readily available light where no emitter of light is shined into the eye. For example, although there may be lamps and light fixtures in a room wherein devices and methods described in this disclosure are practiced, these sources or emitters of light are not used purposefully to illuminate the eye and the source of light is not directed into the eye. The subject can be a human or an animal. While the pupil may appear to be black or very dark in a photograph that does not use a flash, the pixel values do vary in magnitude based on the power of the eye. In the images that are obtained for embodiments of this invention, the information needed to measure the optical distortions of the eye is contained within the pixel values of the first and second color.

Non-relevant reflections from the lens and corneal surface are blocked; else these reflections would otherwise obscure measurement of the light within the pupil. For example, the surface closest to the patient of the apparatus acquiring the images can be matte and black so that it does not create corneal reflections that would obscure the measurement, or a polarizing filter can be used.

Once this image is obtained, the pupil and its border are identified (i.e., segmented). Light within the pupil is then analyzed. No light is shined into the eye. The total intensity of the pupil is used in a formula that calculates the autorefraction result, and a minimum intensity is required, but differences in intensity across the pupil are not measured for autorefraction. The light in an eye with spherical refractive error does not have a slope; it is of uniform intensity within the pupil. Even the difference between the pixels of the first color and the second color is uniform across the pupil for spherical refractive error (i.e., no astigmatism). Ambient light from the room that is always reflecting off of the retina is measured. A difference in the intensity of the first color versus the second color pixel values is determined and compared; this difference is related to the eye's refractive error/glasses prescription. For example, the difference between the first color and second color pixels is a larger number in hyperopia (farsighted) and a lower number in myopia (nearsighted). Also, the light within the pupil of eyes with hyperopia is somewhat brighter than eyes with myopia. In the case of astigmatism, the intensity of individual pixels across the pupil will have a higher standard deviation than with hyperopia or myopia alone. In most eyes, the axis of the astigmatism is known to be regular, meaning that the two principal power meridians are 90 degrees apart. In the present disclosure, the presence of astigmatism within an optical system causes differences in intensity within the pupil. The more myopic meridian will be dimmer and the more hyperopic meridian will be brighter.

Disclosed herein are methods and systems for making a determination about an eye. The eye is aligned with an image acquisition device using a software application executing on the image acquisition device that provides visual and/or audible indicators through peripherals of the image acquisition device. The acquired image(s) is then transmitted to a cloud-based network where a determination about the eye is made based on the image(s). In some instances, a trained person has an ability to access, review, confirm or modify the determination made about the eye.

In each of the disclosed methods and systems, the eye can be examined and/or image acquired using a smart device such as a smart phone, tablet, laptop computer, etc. that has an ability to receive reflections from the eye and analyze them using software executing on a processor and/or transmit or receive data over a network. For example, an image of an eye may be acquired using a camera of a smart phone and the image or data associated with the image transmitted wirelessly to a cloud-based network where the image is further analyzed and the results of the analysis transmitted back to the smart phone. The smart phone may be executing an application (an “app”) that assists with aligning the camera with the eye for proper image acquisition, obtain the image, pre-processing or partially processing the image, transmitting the image to the cloud-based network, receiving results of an analysis of the image or image data from the cloud-based network, and reporting or displaying information associated with the results on the smart phone. Such a process may in some instances comprise a process using telehealth procedures or when used by a layperson in a vision screening.

In other instances, the determination about the eye is the presence and/or severity of a cataract or optical distortion or opacity within the eye.

It should be understood that the above-described subject matter may also be implemented as a computer-controlled apparatus, a computer process, a computing system, or an article of manufacture, such as a computer-readable storage medium.

In one aspect, a method of making a determination about the eye of a subject is disclosed. One embodiment of the method comprises aligning an eye with an image capture device; capturing an image of the eye with the image capture device; transmitting the captured image of the eye to a computing device; detecting, using the computing device, light reflected out of an eye of a subject from a retina of the eye of the subject; and making a determination about the eye of the subject based upon the reflected light. In some instances, the light reflected out of an eye of a subject from a retina of the eye of the subject comprises ambient light. In some instances, the computing device comprises at least a part of a cloud-based network. In some instances, the method further comprises having the determination about the eye of the subject reviewed, confirmed or adjusted by a trained person. In some instances, making a determination about the eye of the subject based upon the reflected light comprises making a determination based at least in part on an aspect of the reflected light. For example, making a determination about the eye of the subject based upon the reflected light may comprise making a determination based at least in part on a brightness and one or more colors of the reflected light.

In some instances of the method, making a determination about the eye of the subject based upon the reflected light comprises making a determination of a refractive error for the eye of the subject based at least in part on an aspect of the reflected light. For example, making a determination about the eye of the subject based upon the reflected light may comprise making a determination of a refractive error for the eye of the subject based at least in part on a brightness and one or more colors of the reflected ambient light.

In some instances of the method, detecting, using the computing device, light reflected out of an eye of a subject from a retina of the eye of the subject further comprises capturing, using the image capture device, the image of the eye of a subject, wherein non-relevant reflections from the eye of the subject are managed while capturing the image; determining, using the computing device, an overall intensity of light from a plurality of pixels located within the at least a portion of a pupil captured in the image; determining, using the computing device, a first intensity of a first color from the plurality of pixels located within the at least a portion of a pupil of the eye of the subject captured in the image; determining, using the computing device, a second intensity of a second color from the plurality of pixels located within the at least a portion of the pupil of the eye of the subject captured in the image; and comparing, by the computing device, a relative intensity of the first color and a relative intensity of the second color, wherein the comparison and said overall intensity are used to make the determination about the eye of the subject based upon the reflected light. In some instances, the first color may comprise any one or any combination of red, green, and blue. In some instances, the second color may comprise any one or any combination of red, green, and blue.

In some instances of the method, the determination about the eye of the subject based upon the reflected ambient light comprises an autorefraction or a photorefraction measurement.

In some instances of the method, capturing, using the image capture device, the image of the eye of the subject comprises capturing a first image with the image capture device through a spectacle lens or a contact lens while the subject is wearing the spectacle lens or the contact lens over the eye and capturing a second image with the image capture device while the subject is not wearing the spectacle lens or the contact lens over the eye and the first image is compared to the second image and the determination about the eye of the subject based upon the reflected light is based on the comparison and comprises an estimated prescription for the spectacle lens or the contact lens.

In some instances of the method, the first intensity of the first color is brighter relative to the second intensity of the second color and the overall intensity is relatively brighter, and the determination about the eye of the subject based upon the reflected light comprises a positive value or hyperopia.

In some instances of the method, first intensity of the first color is dimmer relative to the second intensity of the second color and the overall intensity is relatively dimmer, and the determination about the eye of the subject based upon the reflected light comprises a negative value or myopia.

In some instances, the method further comprises making a first determination about the eye of the subject based upon the reflected light from a first plurality of pixels located within the at least a portion of the pupil of the eye of the subject captured in the image; making a second determination from a second plurality of pixels located within the at least a portion of the pupil of the eye of the subject captured in the image, wherein the second plurality of pixels are a subset of the first plurality of pixels; making a third determination from a third plurality of pixels located within the at least a portion of the pupil of the eye of the subject captured in the image, wherein the third plurality of pixels are a subset of the first plurality of pixels and are separate from the second plurality of pixels; and comparing the first determination, the second determination and the third determination to make the determination about the eye of the subject based upon the reflected light. In some instances, comparing the first determination, the second determination and the third determination to make the determination about the eye of the subject based upon the reflected light comprises one or more of determining a standard deviation of the first determination to the second determination, a standard deviation of the first determination to the second determination, or a standard deviation of the second determination to the third determination, wherein the determined standard deviation indicates the determination about the eye of the subject based upon the reflected light. In some instances, the determination about the eye of the subject based upon the reflected light is a presence or an absence of astigmatism. In some instances, the presence of astigmatism is detected and an amount of astigmatism is determined by comparing the overall intensity and the relative intensity of the first color or the relative intensity of the second color of various regions of the pupil.

In some instances of the method, managing non-relevant reflections from the eye while capturing the image comprises managing reflections from a cornea or a lens of the eye of the subject while capturing the image. In some instances, managing non-relevant reflections from the eye while capturing the image comprises placing a polarizing filter over a lens of the image capture device or between the image capture device and the eye of the subject. In some instances, managing non-relevant reflections from the eye while capturing the image comprises blocking light that would lead to reflections from a corneal surface of the eye or a lens of the eye. In some instances, managing non-relevant reflections from the eye while capturing the image comprise providing a surface that absorbs light or prevents the non-relevant reflections from the eye while capturing the image. For example, the surface may comprise a surface having a black matte finish. In some instances, the surface may comprise a portion of the image capture device. For example, the surface may comprise at least a portion of a case that houses the image capture device.

In some instances of the method, the image capture device comprises a smart phone or other mobile computing device having a camera. In some instances, the eye is aligned with the image capture device using a software application executing on the smart phone or other mobile computing device having a camera. In some instances, the software application executing on the smart phone or other mobile computing device having a camera provides visual and/or audible indicators through peripherals of the smart phone or other mobile computing device having a camera to align the eye with the image capture device. In some instances, the captured image of the eye is transmitted wirelessly to the computing device.

In some instances of the method, the image capture device captures a still image or a video of the eye of the subject.

In some instances of the method, the subject's pupil has a diameter of approximately 2 mm or less. In some instances, the subject's pupil is a natural pupil. In some instances, the subject's pupil is an artificial pupil.

In some instances of the method, the eye of the subject is the subject's left eye or right eye. In some instances, the eye of the subject is the subject's left eye and right eye.

In some instances of the method, the method further comprises detecting an intensity for the ambient light conditions and providing an indication if the ambient light conditions are too low for the image capture device to capture the image of the eye of the subject.

In some instances of the method, the captured image is pre-processed. For example, the captured image is pre-processed prior to transmitting the captured image of the eye to the computing device by a processor associated with the image capture device. In some instances, the captured image is pre-processed by the computing device. In some instances, the captured image is pre-processed by segmenting a sclera in the captured image.

In some instances of the method, a color temperature of the lighting is detected and used to make the determination about the eye of the subject based upon the reflected light, wherein the reflected light is adjusted based on the determined color temperature of the lighting.

In some instances of the method, making the determination about the eye of the subject based upon the reflected light comprises making a determination about a presence and/or severity of a cataract or optical distortion or opacity within the eye.

In another aspect, a method of determining an optical quality of the eye is disclosed. One embodiment of the method comprises aligning an eye of a subject with an image capture device; capturing, using the image capture device, an image of the eye of the subject, wherein non-relevant reflections from a cornea and a lens of the eye of the subject are managed while capturing the image; pre-processing at least a portion of the image of the eye of the subject; transmitting the pre-processed portion of the image of the eye of the subject to a computing device; determining, using the computing device, an overall intensity of light from a plurality of pixels located within at least a portion of a pupil captured in the pre-processed portion of the image, wherein the plurality of pixels comprise red, green, and blue pixels; determining, using the computing device, an average red intensity from the plurality of pixels located within the at least a portion of the pupil captured in the pre-processed portion of the image; determining, using the computing device, an average blue intensity from the plurality of pixels located within the at least a portion of a pupil captured in the pre-processed portion of the image; and determining, by the computing device, using the average red intensity, the average blue intensity and the determined overall intensity an optical quality of the eye.

In some instances of the method, the image of the eye is captured using only ambient lighting.

In some instances of the method, the determined optical quality of the eye comprises an autorefraction or photorefraction measurement.

In some instances of the method, capturing, using the image capture device, the image of the eye of the subject comprises capturing a first image with the image capture device through a spectacle lens or a contact lens while the subject is wearing the spectacle lens or the contact lens over the eye and capturing a second image with the image capture device while the subject is not wearing the spectacle lens or the contact lens over the eye and the first image is compared to the second image and the determined optical quality of the eye is based on the comparison and comprises an estimated prescription for the spectacle lens or the contact lens.

In some instances of the method, the average red intensity is brighter relative to the average blue intensity and the overall intensity is relatively brighter, and the determined optical quality of the eye is a positive value or hyperopia. In some instances, the average red intensity is dimmer relative to the average blue intensity and the overall intensity is relatively dimmer, and the determined optical quality of the eye is a negative value or myopia.

In some instances the method further comprises making a first determined optical quality about the eye of the subject based upon the reflected light from a first plurality of pixels located within the at least a portion of the pupil of the eye of the subject captured in the pre-processed portion of the image; making a second determined optical quality about the eye from a second plurality of pixels located within the at least a portion of the pupil of the eye of the subject captured in the pre-processed portion of the image, wherein the second plurality of pixels are a subset of the first plurality of pixels; making a third determined optical quality about the eye from a third plurality of pixels located within the at least a portion of the pupil of the eye of the subject captured in the pre-processed portion of the image, wherein the third plurality of pixels are a subset of the first plurality of pixels and are separate from the second plurality of pixels; and comparing the first determined optical quality, the second determined optical quality and the third determined optical quality to make the determined optical quality about the eye of the subject based upon the reflected light. In some instances, comparing the determined optical quality, the second determined optical quality and the third determined optical quality to make the determination about the eye of the subject based upon the reflected light comprises one or more of determining a standard deviation of the first determination to the second determination, a standard deviation of the first determination to the second determined optical quality, or a standard deviation of the second determined optical quality to the third determined optical quality, wherein the determined standard deviation indicates the determined optical quality about the eye of the subject based upon the reflected light.

In some instances of the method, the determined optical quality of the eye is a presence or an absence of astigmatism. In some instances, the presence of astigmatism is detected and an amount of astigmatism is determined by comparing the overall intensity and the average red intensity or the average blue intensity of various regions of the pupil. In some instances, the amount of astigmatism is determined by measuring one or more of hyperopia or myopia at the various regions of the pupil.

In some instances of the method, managing non-relevant reflections from the cornea and the lens of the eye of the subject while capturing the image comprises placing a polarizing filter over a lens of the image capture device or between the image capture device and the eye of the subject.

In some instances of the method, managing non-relevant reflections from the cornea and the lens of the eye of the subject while capturing the image comprises blocking light that would lead to reflections from a corneal surface or the lens of the eye. In some instances, the image capture device further comprises a surface having a black matte finish and blocking light that would lead to reflections from a corneal surface or the lens of the eye comprises the surface absorbing light or preventing reflections from the corneal surface or the lens of the eye caused by the lighting conditions. In some instances, the surface comprises at least a portion of a case that houses the image capture device.

In some instances of the method, the image capture device comprises a smart phone or other mobile computing device having a camera. In some instances, the eye is aligned with the image capture device using a software application executing on the smart phone or other mobile computing device having a camera. In some instances, the software application executing on the smart phone or other mobile computing device having a camera provides visual and/or audible indicators through peripherals of the smart phone or other mobile computing device having a camera to align the eye with the image capture device.

In some instances of the method, the computing device comprises at least a portion of a cloud-based network. In some instances, the captured image of the eye is transmitted wirelessly to the computing device.

In some instances of the method, the image capture device captures a still image or a video of the eye of the subject.

In some instances of the method, the subject's pupil has a diameter of approximately 2 mm or less.

In some instances of the method, the subject's pupil is a natural pupil. In some instances, the subject's pupil is an artificial pupil.

In some instances of the method, the eye of the subject is the subject's left eye or right eye. In some instances, the eye of the subject is the subject's left eye and right eye.

In some instances of the method, the method further comprises detecting an intensity for the ambient light conditions and providing an indication if the ambient light conditions are too low for the image capture device to capture the image of the eye of the subject.

In some instances of the method, pre-processing the at least the portion of the image of the eye of the subject comprises segmenting a sclera in the image of the eye of the subject.

In some instances of the method, the determined optical quality of the eye reviewed, confirmed or adjusted by a trained person.

In some instances of the method, a color temperature of the lighting is detected and used to make the determination about the eye of the subject based upon the reflected light, wherein the reflected light is adjusted based on the determined color temperature of the lighting.

In some instances of the method, the determined optical quality of the eye is a presence and/or severity of a cataract or optical distortion or opacity within the eye.

In another aspect, a system for making a determination about the eye of the subject based upon the detected reflected light is disclosed. The system is comprised of an apparatus. The apparatus is comprised of an image capture device; a memory; a network interface; and a processor in communication with the memory, the image capture device, and the network interface, wherein the processor executes computer-readable instructions stored in the memory that cause the processor to assist in aligning an eye of a subject with the image capture device; capture, using the image capture device, an image of an eye of a subject, wherein non-relevant reflections from the eye of the subject are managed while capturing the image; pre-process the captured image of the eye of the subject; and, transmit, using the network interface, the pre-processed captured image of the eye of the subject to a computing device, wherein a processor of the computing device executes computer-readable instructions stored in a memory of the computing device that cause the processor of the computing device to detect, from the pre-processed image of the eye of the subject, light reflected out of the eye of a subject from a retina of the eye of the subject; and make a determination about the eye of the subject based upon the detected reflected light. In some instances of the system, the reflected light comprises reflected ambient light.

In some instances of the system, the processor of the computing device executing computer-readable instructions stored in the memory of the computing device that cause the processor of the computing device to make a determination about the eye of the subject based upon the detected reflected light comprises the processor of the computing device executing computer-readable instructions stored in the memory of the computing device that cause the processor of the computing device to make a determination based at least in part on an aspect of the reflected light.

In some instances of the system, the processor of the computing device executing computer-readable instructions stored in the memory of the computing device that cause the processor of the computing device to make a determination about the eye of the subject based upon the detected reflected light comprises the processor of the computing device executing computer-readable instructions stored in the memory of the computing device that cause the processor of the computing device to make a determination based at least in part on a brightness and one or more colors of the reflected light.

In some instances of the system, the processor of the computing device executing computer-readable instructions stored in the memory of the computing device that cause the processor of the computing device to make a determination about the eye of the subject based upon the detected reflected light comprises the processor of the computing device executing computer-readable instructions stored in the memory of the computing device that cause the processor of the computing device to make a determination of a refractive error for the eye of the subject based at least in part on an aspect of the reflected light.

In some instances of the system, the processor of the computing device executing computer-readable instructions stored in the memory of the computing device that cause the processor of the computing device to make a determination about the eye of the subject based upon the detected reflected ambient light comprises the processor of the computing device executing computer-readable instructions stored in the memory of the computing device that cause the processor of the computing device to make a determination of a refractive error for the eye of the subject based at least in part on a brightness and one or more colors of the reflected ambient light.

In some instances of the system, the processor of the computing device executing computer-readable instructions stored in the memory of the computing device that cause the processor of the computing device to detect light reflected out of an eye of a subject from a retina of the eye of the subject further comprises the processor of the computing device executing computer-readable instructions stored in the memory of the computing device that cause the processor of the computing device to determine an overall intensity of light from a plurality of pixels located within the at least a portion of a pupil captured in the pre-processed image; determine a first intensity of a first color from the plurality of pixels located within the at least a portion of a pupil of the eye of the subject captured in the pre-processed image; determine a second intensity of a second color from the plurality of pixels located within the at least a portion of the pupil of the eye of the subject captured in the pre-processed image; and determine using a relative intensity of the first color and a relative intensity of the second color and the overall intensity the determination about the eye of the subject based upon the reflected light. In some instances, the first color comprises any one or any combination of red, green, and blue. In some instances, the second color comprises any one or any combination of red, green, and blue.

In some instances of the system, the processor of the computing device executing computer-readable instructions stored in the memory of the computing device that cause the processor of the computing device to make a determination about the eye of the subject based upon the detected reflected light comprises the processor of the computing device executing computer-readable instructions stored in the memory of the computing device that cause the processor of the computing device to make an autorefraction or a photorefraction measurement.

In some instances of the system, capturing, using the image capture device, the image of the eye of the subject comprises capturing a first image with the image capture device through a spectacle lens or a contact lens while the subject is wearing the spectacle lens or the contact lens over the eye and capturing a second image with the image capture device while the subject is not wearing the spectacle lens or the contact lens over the eye the processor of the computing device executing computer-readable instructions stored in the memory of the computing device that cause the processor of the computing device to compare the first image to the second image and the determination about the eye of the subject based upon the reflected light is based on the comparison and comprises an estimated prescription for the spectacle lens or the contact lens.

In some instances of the system, the first intensity is brighter relative to the second intensity and the overall intensity is relatively brighter, and the determination about the eye of the subject based upon the reflected light comprises a positive value or hyperopia.

In some instances of the system, the first intensity is dimmer relative to the second intensity and the overall intensity is relatively dimmer, and the determination about the eye of the subject based upon the reflected ambient comprises a negative value or myopia.

In some instances of the system, the processor of the computing device executing computer-readable instructions stored in the memory of the computing device that cause the processor of the computing device to make a determination about the eye of the subject based upon the detected reflected light comprises the processor of the computing device executing computer-readable instructions stored in the memory of the computing device that cause the processor of the computing device to make a first determination about the eye of the subject based upon the reflected light from a first plurality of pixels located within the at least a portion of the pupil of the eye of the subject captured in the pre-processed image; make a second determination from a second plurality of pixels located within the at least a portion of the pupil of the eye of the subject captured in the pre-processed image, wherein the second plurality of pixels are a subset of the first plurality of pixels; make a third determination from a third plurality of pixels located within the at least a portion of the pupil of the eye of the subject captured in the pre-processed image, wherein the third plurality of pixels are a subset of the first plurality of pixels and are separate from the second plurality of pixels; and compare the first determination, the second determination and the third determination to make the determination about the eye of the subject based upon the reflected light. For example, in some instances the processor of the computing device executing computer-readable instructions stored in the memory of the computing device that cause the processor of the computing device to make a determination about the eye of the subject based upon the detected reflected light comprises the processor of the computing device executing computer-readable instructions stored in the memory of the computing device that cause the processor of the computing device to compare the first determination, the second determination and the third determination to make the determination about the eye of the subject based upon the reflected light comprises one or more of determining a standard deviation of the first determination to the second determination, a standard deviation of the first determination to the second determination, or a standard deviation of the second determination to the third determination, wherein the determined standard deviation indicates the determination about the eye of the subject based upon the reflected light. In some instances, the determination about the eye of the subject based upon the reflected light is a presence or an absence of astigmatism. In some instances, the presence of astigmatism is detected and an amount of astigmatism is determined by comparing the overall intensity and the relative intensity of the first color or the relative intensity of the second color of various regions of the pupil. In some instances, the amount of astigmatism is determined by measuring one or more of hyperopia or myopia at the various regions of the pupil.

In some instances of the system, the image capture device is configured to manage non-relevant reflections from a cornea and a lens of the eye of the subject while capturing the image.

In some instances, the system further comprises a polarizing filter, wherein non-relevant reflections from the eye are managed while capturing the image by placing the polarizing filter over a lens of the image capture device or between the image capture device and the eye of the subject when capturing the image.

In some instances, the system further comprises a surface, wherein managing non-relevant reflections from the eye while capturing the image comprises the surface absorbing light or preventing the non-relevant reflections from the eye while capturing the image. In some instances, the surface comprises a surface having a black matte finish. In some instances, the surface comprises a portion of the image capture device. In some instances, the surface comprises at least a portion of a case that houses the image capture device.

In some instances of the system, the image capture device comprises at least a portion of a smart phone or other mobile computing device having a camera. In some instances, the eye is aligned with the image capture device using a software application executing on the smart phone or other mobile computing device having a camera. In some instances, the software application executing on the smart phone or other mobile computing device having a camera provides visual and/or audible indicators through peripherals of the smart phone or other mobile computing device having a camera to align the eye with the image capture device.

In some instances of the system, the computing device comprises at least a portion of a cloud-based network. In some instances, the captured pre-processed image of the eye is transmitted wirelessly to the computing device. In some instances, the system further comprises a separate computing device configured to access the cloud-based network, wherein the separate computing device is used by a trained person to review, confirm or adjust the determination about the eye of the subject.

In some instances of the system, the image capture device captures a still image or a video of the eye of the subject.

In some instances of the system, the portion of the pupil of the eye of the subject comprises a pupil having a diameter of approximately 2 mm or less. In some instances, the subject's pupil is a natural pupil. In some instances, the subject's pupil is an artificial pupil.

In some instances of the system, the eye of the subject is the subject's left eye or right eye. In some instances, the eye of the subject is the subject's left eye and right eye.

In some instances, the system further comprises a light meter, wherein the light meter detects an intensity for the ambient light conditions and provides an indication if the ambient light conditions are too low for the image capture device to capture the image of the eye of the subject.

In some instances of the system, pre-processing the at least the portion of the image of the eye of the subject comprises segmenting a sclera in the image of the eye of the subject.

In some instances, the system further comprises a light meter, wherein a color temperature of the light is detected by the light meter and used to make the determination about the eye of the subject based upon the reflected light, wherein the reflected light is adjusted based on the determined color temperature of the lighting.

In some instances of the system, making the determination about the eye of the subject based upon the detected reflected light comprises making a determination about a presence and/or severity of a cataract or optical distortion or opacity within the eye.

Yet another aspect comprises a method of making a determination about retinal nerve fiber health of an eye. One embodiment of the method comprises positioning a polarizing filter on an image sensor; aligning the image sensor and polarizing filter with the eye; orienting the polarizing filter and image sensor to a first position; capturing, using the image sensor, a first image of the eye while the polarizing filter and image sensor are oriented in the first position, wherein said captured image includes a retina of the eye; orienting the polarizing filter and image sensor to a second position, wherein the second position is offset by 90 degrees from the first position; capturing, using the image sensor, a second image of the eye, wherein the second image also includes the retina; determining a first brightness value for a first region of the first image that corresponds to the retina; determining a second brightness value for a second region of the second image that corresponds to the retina; and determining a score based on at least the first brightness value and the second brightness value, wherein the score represents health of the retina.

In some instances of the method, the image sensor comprises a part of a smart device such as a smart phone, tablet, laptop computer, and the like, and the image sensor is an image acquisition device associated with the smart device.

In some instances of the method, aligning the image sensor and polarizing filter with the eye comprises using a software application executing on the smart device. In some instances, the software application executing on the smart device provides visual and/or audible indicators through peripherals of the smart device to align the eye with the image sensor and polarizing filter. In some instances of the method, orienting the polarizing filter and image sensor to the second position comprises rotating the smart device by 90 degrees and re-aligning the image sensor and polarizing filter with the eye. In some instances the software application executing on the smart device instructs a user how to orient the smart device for capturing the second image by providing visual and/or audible indicators of proper orientation through peripherals of the smart device.

In some instances of the method, the first image of the eye and the second image of the eye are captured by the image sensor of the smart device and the captured first image of the eye and the captured second image of the ere are transmitted wirelessly to a computing device, wherein the computing device performs the steps of determining the first brightness value for the first region of the first image that corresponds to the retina; determining the second brightness value for the second region of the second image that corresponds to the retina; and determining the score based on at least the first brightness value and the second brightness value, wherein the score represents health of the retina. In some instances, the computing device comprises at least part of a cloud-based network.

In some instances of the method, a trained person can access, review, confirm or adjust the determined score about the health of the retina.

In another aspect, a method for automatically measuring a subject's phoria while the subject fixates on a visual target is disclosed. One embodiment of the method comprises aligning an eye with an image capture device; capturing an image of at least one of the subject's eyes using the image capturing device, the image including a reflection of light from any surface of the at least one of the subject's eyes; transmitting the captured image of the at least one of the subject's eyes to a computing device; analyzing the image to identify a position of the reflection of the light within the at least one of the subject's eyes; and determining a phoria measurement based on the position of the reflection of the light within the at least one of the subject's eyes.

In some instances, the method further comprises comparing a position of the reflection of the light within one of the subject's eyes and a position of the reflection of the light within another one of the subject's eyes, wherein the phoria measurement is determined based on a result of the comparison.

In some instances of the method, analyzing the image to identify a position of the reflection of the light within the at least one of the subject's eyes further comprises identifying a position of the reflection of the light relative to a landmark of the at least one of the subject's eyes. In some instances, the image includes a reflection of the light from at least one of an outer or inner surface of a cornea or an outer or inner surface of a lens of the at least one of the subject's eyes. In some instances, the image is a first, second, third or fourth Purkinje image.

In some instances, the method further comprises covering and uncovering the at least one of the subject's eyes, wherein the eye is aligned with the image capture device and the image is captured after uncovering the at least one of the subject's eyes. In some instances, the method further comprises capturing a sequence of images of the subject's eyes after aligning the eye with the image capture device and uncovering the at least one of the subject's eyes, and comparing a position of the reflection of the light within the at least one of the subject's eyes in one of the sequence of images to a position of the reflection of the light within the at least one of the subject's eyes in another of the sequence of images to determine any a magnitude and a direction of any movement after the at least one of the subject's eyes is uncovered.

In some instances, the method further comprises covering at least one of the subject's eyes with a filter, wherein the image is captured while at least one of the subject's eyes is covered by the filter.

In some instances, the method further comprises performing an autorefraction measurement, the autorefraction measurement measuring a power of one of the subject's eyes while focusing on the visual target. In some instances, the image is captured in response to the power of the one of the subject's eyes being within a predetermined range. In some instances, the method further comprises adjusting the phoria measurement based on the autorefraction measurement. In some instances, the method further comprises calculating an accommodative convergence accommodation ratio based on a position of the reflection of the light within at least one of the subject's eyes and the autorefraction measurement.

In some instances of the method, the at least one of the subject's eyes is the subject's left eye or right eye. In some instances, the at least one of the subject's eyes is the subject's left eye and right eye.

In some instances, the method further comprises illuminating at least one of the subject's eyes with a light using a light source to create the reflection.

In some instances of the method, the light is in a visible or non-visible portion of an electromagnetic spectrum.

Yet another aspect comprises a system for automatically measuring a subject's phoria while the subject fixates on a visual target. One embodiment of the system comprises an apparatus comprised of an image capture device; a memory; a network interface; and a processor in communication with the memory, the image capture device, and the network interface, wherein the processor executes computer-readable instructions stored in the memory that cause the processor to assist in aligning an eye of a subject with the image capture device; capture, using the image capture device, an image of an eye of a subject while focusing on the visual target; and transmit, using the network interface, the captured image of the eye of the subject to a computing device, wherein a processor of the computing device executes computer-readable instructions stored in a memory of the computing device that cause the processor of the computing device to perform an autorefraction measurement that measures a power of at least one of the subject's eyes based on the captured image; analyze the image to identify a position of the reflection of the light within the at least one of the subject's eyes; and determine a phoria measurement based on the position of the reflection of the light within the at least one of the subject's eyes.

In some instances, the memory of the computing device has further computer-executable instructions stored thereon that, when executed by the processor, cause the processor of the computing device to compare a position of the reflection of the light within one of the subject's eyes and a position of the reflection of the light within another one the subject's eyes, wherein the phoria measurement is determined based on a result of the comparison.

In some instances of the system, analyzing the image to identify a position of the reflection of the light within at least one of the subject's eyes further comprises identifying a position of the reflection of the light relative to a landmark of the at least one of the subject's eyes.

In some instances of the system, the image includes a reflection of the light from at least one of an outer or inner surface of a cornea or an outer or inner surface of a lens of at least one of the subject's eyes. In some instances, the image is a first, second, third or fourth Purkinje image.

In some instances of the system, the image is captured after covering and uncovering the at least one of the subject's eyes. In some instances, the image capture device is a video capturing device or a camera for capturing a sequence of images of the subject's eyes after uncovering the at least one of the subject's eyes and wherein the processor of the computing device executes computer readable instructions to compare a position of the reflection of the light within the at least one of the subject's eyes in one of the sequence of images to a position of the reflection of the light within the at least one of the subject's eyes in another of the sequence of images to determine a magnitude and a direction any movement after the at least one of the subject's eyes is uncovered.

In some instances of the system, the image is captured while at least one of the subject's eyes is covered by a filter.

In some instances, the system further comprises a display device, wherein the apparatus defines a first surface and a second surface opposite to the first surface, the display device being arranged on the first surface and the image capturing device being arranged on the second surface.

In some instances, the system further comprises a light source for illuminating the at least one of the subject's eyes with a light. In some instances, the light source comprises a plurality of LEDs arranged around the image capture device.

In some instances of the system, the apparatus provides the visual target for the subject, and the memory has further computer-executable instructions stored thereon that, when executed by the processor, cause the processor of the apparatus to perform an autorefraction measurement that measures a power of the one of the subject's eyes while focusing on the visual target.

In some instances of the system, the image is captured in response to the power of the one of the subject's eyes being within a predetermined range.

In some instances of the system, the memory of the computing device has further computer-executable instructions stored thereon that, when executed by the processor of the computing device, cause the processor of the computing device to adjust the phoria measurement based on the autorefraction measurement.

In some instances of the system, the memory of the computing device has further computer-executable instructions stored thereon that, when executed by the processor of the computing device, cause the processor of the computing device to calculate an accommodative convergence accommodation ratio based on a position of the reflection of the light within at least one of the subject's eyes and the autorefraction measurement.

In some instances of the system, the computing device is at least a part of a cloud-based network.

In some instances of the system, the light is in a visible or non-visible portion of an electromagnetic spectrum.

Another aspect comprises a method for automatically measuring alignment of at least one of a subject's eyes. One embodiment of the method comprises aligning a subject's eyes with an image capture device; performing an autorefraction measurement, the autorefraction measurement measuring a power of at least one of the subject's eyes while focusing on a visual target; capturing an image of the subject's eyes using the image capture device, the image including a reflection of light from any surface of each of the subject's eyes; transmitting the captured image and the autorefraction measurement to a computing device; analyzing the image using the computing device to identify a position of the reflection of the light within each of the subject's eyes, respectively; and determining, by the computing device, an alignment measurement of at least one of the subject's eyes based on the position of the reflection of the light within each of the subject's eyes, respectively.

In some instances of the method, the image is captured in response to the power of at least one of the subject's eyes being within a predetermined range. In some instances, the method further comprises adjusting the alignment measurement of at least one of the subject's eyes based on the autorefraction measurement.

In some instances, the method further comprises calculating an accommodative convergence accommodation ratio based on a position of the reflection of the light within the at least one of the subject's eyes and the autorefraction measurement.

In some instances, the method further comprises comparing a position of the reflection of the light within one of the subject's eyes and a position of the reflection of the light within another one of the subject's eyes, wherein the alignment measurement is determined based on a result of the comparison.

In some instances of the method, analyzing the image to identify a position of the reflection of the light within each of the subject's eyes, respectively, further comprises identifying a position of the reflection of the light relative to a visible portion of an iris of each of the subject's eyes, respectively.

In some instances of the method, the image includes a reflection of the light from at least one of an outer or inner surface of a cornea or an outer or inner surface of a lens of at least one of the subject's eyes. In some instances, the image is a first, second, third or fourth Purkinje image.

In some instances of the method, the alignment measurement is a phoria measurement or a tropia measurement.

In some instances of the method, the at least one of the subject's eyes is the subject's left eye or right eye. In some instances, the at least one of the subject's eyes is the subject's left eye and right eye.

In some instances of the method, the light is in a visible or non-visible portion of an electromagnetic spectrum.

In some instances, the method further comprises illuminating the subject's eyes with a light to create the reflection.

In another aspect, a method for measuring alignment of at least one eye is disclosed. One embodiment of the method comprises aligning at least one of a subject's eyes with an image capture device; capturing an image of the at least one of a subject's eyes; transmitting the captured image to a computing device; performing, by the computing device, an autorefraction measurement based on the captured image of the at least one of a subject's eyes; performing, by the computing device, an alignment measurement of the at least one of the subject's eyes based on the captured image of the at least one of a subject's eyes; and compensating, by the computing device, the alignment measurement based on the autorefraction measurement. In some instances, the alignment measurement is a phoria measurement or a tropia measurement.

In another aspect, a method for automatically measuring a subject's phoria while the subject fixates on a visual target is disclosed. In one embodiment, the method comprises aligning at least one of a subject's eyes with an image capture device; capturing an image of at least one of the subject's eyes using the image capture device, the image including at least two reflections of light from at least one of the subject's eyes; transmitting the captured image to a computing device; analyzing, by the computing device, the image to identify respective positions of the at least two reflections of the light within the at least one of the subject's eyes; and determining, by the computing device, a phoria measurement based on the respective positions of the at least two reflections of the light within the at least one of the subject's eyes.

In some instances, the method further comprises comparing respective positions of the at least two reflections of the light within one of the subject's eyes and respective positions of the at least two reflections of the light within another one the subject's eyes, wherein the phoria measurement is determined based on a result of the comparison.

In some instances of the method, the image includes at least two reflections of the light from at least two of an outer or inner surface of a cornea or an outer or inner surface of a lens of at least one of the subject's eyes.

In some instances, the method further comprises illuminating at least one of the subject's eyes with a light using a light source to create the reflection.

In another aspect, a method for automatically measuring a subject's phoria while the subject fixates on a visual target is disclosed. One embodiment of the method comprises aligning at least one of a subject's eyes with an image capture device; illuminating the at least one of the subject's eyes with at least two lights using at least two light sources; capturing an image of at least one of the subject's eyes using the image capture device, the image including reflections of the at least two lights from at least one of the subject's eyes; transmitting the image to a computing device; analyzing, by the computing device, the image to identify respective positions of the reflections of the at least two lights within the at least one of the subject's eyes; and determining, by the computing device, a phoria measurement based on the respective positions of the reflections of the at least two lights within the at least one of the subject's eyes.

In some instances, the method further comprises comparing respective positions of the reflections of the at least two lights within one of the subject's eyes and respective positions of the reflections of the at least two lights within another one the subject's eyes, wherein the phoria measurement is determined based on a result of the comparison.

In some instances of the method, the image includes reflections of the at least two lights from at least one of an outer or inner surface of a cornea or an outer or inner surface of a lens of the at least one of the subject's eyes.

In another aspect, a method for automatically measuring a subject's phoria while the subject fixates on a visual target is disclosed. In one embodiment, the method comprises aligning at least one of a subject's eyes with an image capture device; capturing an image of at least one of the subject's eyes using the image capture device, the image including a landmark within at least one of the subject's eyes; transmitting the image to a computing device; analyzing, by the computing device, the image to identify a position of the landmark within the at least one of the subject's eyes; and determining, by the computing device, a phoria measurement based on the position of the landmark within the at least one of the subject's eyes. In some instances, the landmark is a feature within the at least one of the subject's eyes. For example, the feature may be a blood vessel.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A illustrates a flowchart of an exemplary method for making a determination about an eye using one or more images of an eye.

FIG. 1B illustrates an exemplary system for performing telehealth procedures or when used by a layperson in a vision screening;

FIG. 1C illustrates an exemplary overview apparatus for making a determination about the eye of a subject in ambient lighting conditions;

FIGS. 1D-1L illustrate screens of an exemplary app for aligning an eye with an image capture device and acquiring an image of the eye;

FIG. 2A illustrates an example of an apparatus for capturing an image of the eye and making a determination about an eye in ambient lighting conditions;

FIG. 2B illustrates an image of the eye captured by an apparatus for capturing an image of the eye and making a determination about an eye in ambient lighting conditions;

FIG. 2C illustrates an example of an apparatus for capturing an image of the eye and making a determination about an eye in ambient lighting conditions;

FIG. 2D illustrates an image of an eye that can be used to make a determination about the eye such as astigmatism;

FIG. 2E illustrates an example of an apparatus for capturing an image of the eye using polarizing filters and making a determination about an eye in ambient lighting conditions;

FIG. 2F illustrates an example of an apparatus for capturing an image of the eye using a surface and making a determination about an eye in ambient lighting conditions;

FIG. 3 illustrates an example computing device upon which embodiments of the invention may be implemented;

FIG. 4 illustrates an example method for making a determination about an eye of a subject based upon ambient light reflected out of the eye;

FIG. 5 illustrates an alternate example method for making a determination about an eye of a subject based upon ambient light reflected out of the eye;

FIG. 6 is a flowchart for a method of making a determination about an eye of a subject based upon ambient light reflected out of the eye where the determination is made based on ambient light adjusted for color temperature;

FIG. 7 is a flowchart illustrating an exemplary process for making a determination about an eye where the determination comprises measuring retinal nerve fiber layer integrity;

FIGS. 8A-8E illustrate an example automated test for phoria measurement;

FIG. 9 illustrates an example flowchart for a method for automatically measuring a subject's phoria while the subject fixates on a visual target;

FIG. 10 illustrates a flowchart for an example method for automatically measuring alignment of at least one of a subject's eyes;

FIG. 11 illustrates a flowchart of another example method for measuring alignment of at least one eye;

FIG. 12 illustrates a flowchart of another example method for automatically measuring a subject's phoria while the subject fixates on a visual target;

FIG. 13 illustrates a flowchart of yet another example method for automatically measuring a subject's phoria while the subject fixates on a visual target; and

FIG. 14 illustrates a flowchart of another example method for automatically measuring a subject's phoria while the subject fixates on a visual target.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure.

As used in the specification and the appended claims, the singular forms “a,” “an” and “the” include plural referents unless the context clearly dictates otherwise. For example, a referral to an eye includes reference to more than one eye or eyes. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

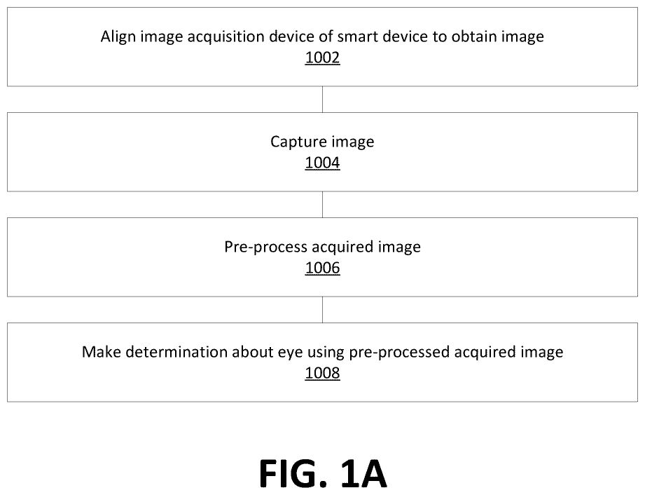

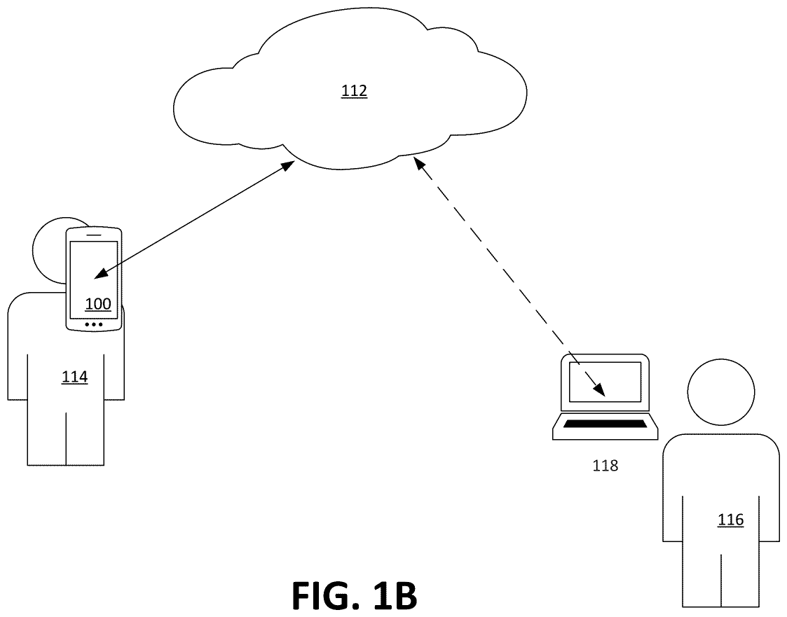

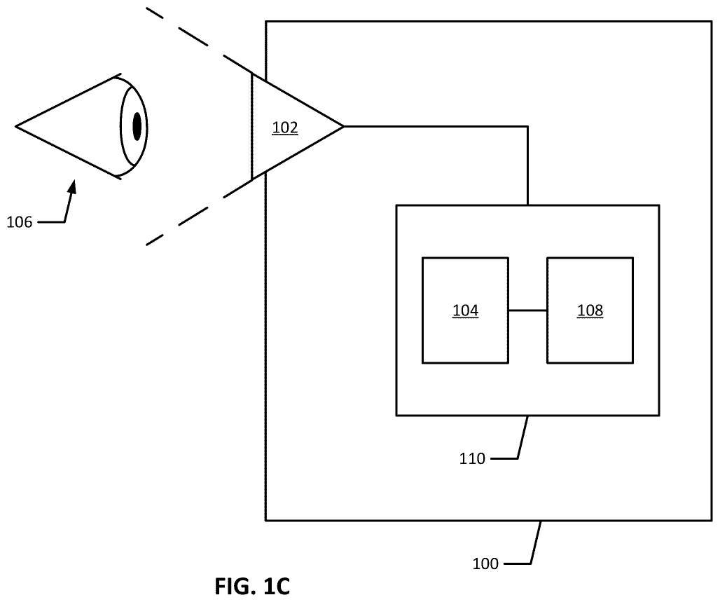

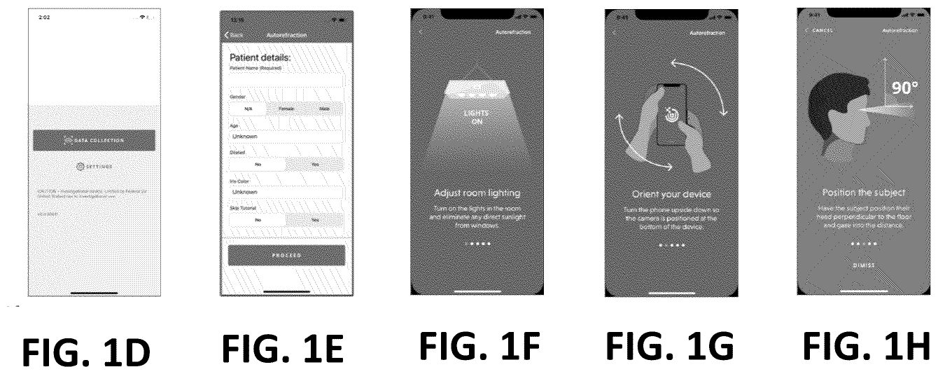

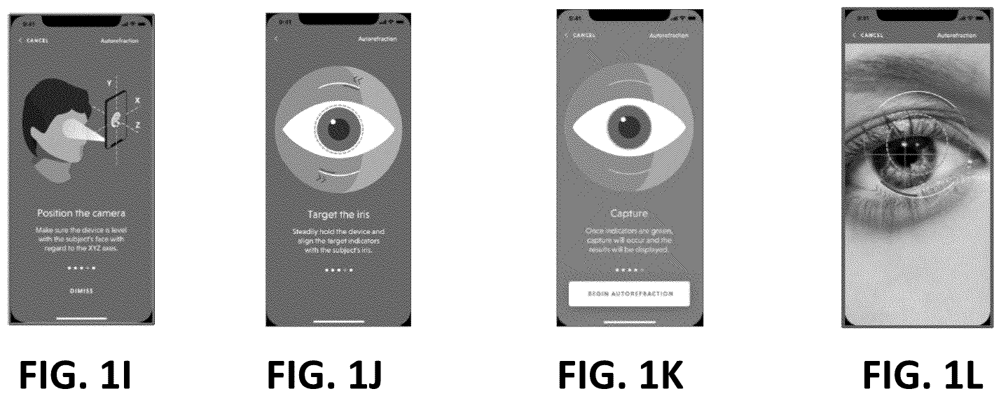

FIG. 1A illustrates a flowchart of an exemplary method for making a determination about an eye using one or more images of an eye. At step 1002, an image acquisition device (e.g., a smart phone camera) is aligned with the eye to obtain an image. At step 1004, the image is acquired. At step 1006, the acquired image is pre-processed. And, at step 1008, the pre-processed image is used to make a determination about the eye. Below, systems for implementing this method are described in greater detail.

FIG. 1B illustrates an exemplary system for performing telehealth procedures or when used by a layperson in a vision screening. As shown in FIG. 1B, a smart device 100 that is configured to wirelessly communicate with a network such as cloud-based network 112 is used to analyze an eye 106 of a person 114. An app executing on the smart device 100 may assist a user of the smart device in aligning a camera of the smart device 100 with the person's eye 106 and acquiring information from the eye 106 including acquiring an image of the eye 106. Once an image and/or other data about the eye 106 is acquired, at least some of the image may be wirelessly transmitted to a network, such as the cloud-based network. In some instances, the transmitted data and/or image is pre-processed by the smart device 100. In other instances, raw data is sent to the cloud-based network 112, where it is pre-processed and/or processed. In the cloud network 112, a determination is made about the eye, as described further herein. The determination may be reviewed and/or approved by a trained person 116 such as a medical specialist (e.g., optometrist, ophthalmologist, etc.). Such a person 116 may access the cloud-based network 112 using a separate computing device 118.

FIG. 1C illustrates an exemplary overview apparatus for making a determination about the eye of a subject. As shown in FIG. 1C, one embodiment of the apparatus 100 comprises an image capture mechanism 102. In one aspect, the image capture mechanism 102 can be camera. The image capture mechanism 102 can take still and/or video images. Generally, the image capture mechanism 102 will be a digital camera, but can be an analog device equipped with or in communication with an appropriate analog/digital converter. The image capture mechanism 102 may also be a webcam, scanner, recorder, or any other device capable of capturing a still image or a video.

In one aspect, apparatus 100 further comprises a computing device 110. In some instances, the image capture mechanism 102 is in direct communication with a computing device 110 through, for example, a network (wired (including fiber optic), wireless or a combination of wired and wireless) or a direct-connect cable (e.g., using a universal serial bus (USB) connection, IEEE 1394 “Firewire” connections, and the like). The computing device 110 may further comprise an interface (not shown). The interface allows the computing device 110 (and apparatus 100) to communicate wirelessly with a cloud-based network 112. In some instances, the image capture mechanism 102 is capable of capturing an image and storing it on a memory device such that the image can be downloaded or transferred to the cloud-based network 112 using, for example, a portable memory device and the like. In one aspect, the computing device 110 and the image capture mechanism 102 can comprise or be a part of a device such as a smart phone, table, laptop computer or any other mobile computing device.

In a basic configuration, the computing device 110 can be comprised of a processor 104 and a memory 108. The processor 104 can execute computer-readable instructions that are stored in the memory 108. Moreover, images captured by the image capture device 102, whether still images or video, can be stored in the memory 108 and processed (or pre-processed) by the processor 104 using computer-readable instructions stored in the memory 108.

The processor 104 is in communication with the image capture device 102 and the memory 108. The processor 104 can execute computer-readable instructions stored on the memory 108 to capture, using the image capture device 102, an image of an eye 106 of a subject. In some instances, no light source, other than ambient lighting, is required to capture the image. The image is captured using only ambient lighting conditions and does not require an additional light source to be directed into the eye 106. While capturing the image of the eye 106, non-relevant reflections from the eye 106 of the subject are managed.

Prior to capturing the image of the eye 106, the image capture device 102 is properly aligned with the eye 106. This may be done using a software application (i.e., “app”) executing stored in the memory 108 and executing on the processor 104. FIGS. 1D-1L illustrate screens of an exemplary app for aligning an eye 106 with an image capture device 102 and acquiring an image of the eye 106. FIG. 1D is an exemplary screen showing opening the app of the apparatus 100 and placing it in data collection mode. FIG. 1E is an image of a patient detail screen so that acquired data can be matched with the patient. In FIG. 1F, a screen is shown where instructions are provided to the user for acquiring the image. For example, the user may be instructed to turn on or off the lights, open or close window shades, and the like. In some instances, the app may be able to use the image acquisition device 102 to determine if a required threshold of light is present, and/or if there is too much light. In FIG. 1G, a screen is shown where additional instructions are provided to the user in regard to orienting the apparatus 100 for image capture. These instructions may be specific to the apparatus 100 being used. For example, if the apparatus 100 is a smart phone, the user may be instructed to orient the smart phone with the image acquisition device 102 (e.g., camera) toward the floor/ground (i.e., positioned at the bottom of the device). At FIG. 1H, a screen is shown where the patient and/or the user is provided additional instructions. For example, the patient may be instructed to gaze at a distant target. Furthermore, the user may be instructed to stand to the side of the subject so as not to obstruct their gaze. FIG. 1I illustrates an exemplary screen of the app where instructions are provided to the user to position the image acquisition device 102. For example, the user may be instructed to make sure the apparatus 100 is vertical and substantially parallel and level with the patient's face in the x, y and z, directions (planes). FIG. 1J is a screen of the app where the eye being imaged is shown positioned relative the image acquisition device 102. The user is instructed to position the image acquisition device in front of the eye 106. Align the circle over the iris, while indicators (e.g., red arrows) prompt the user to rotate or tilt the apparatus 100. Additional indicators (e.g., text) on the screen and verbal commands advise the user to move the apparatus 100 closer to or further back from the eye 106. FIG. 1K is a screen that indicates image capture using the app. In some instances, once the image acquisition device 102 is aligned, the app will auto capture several images. In other instances, the app may operate by voice command. For example, once the image acquisition device 102 is aligned, the user may be able to say, “Go,” “Start,” etc. to capture the images. The above steps/screens can be duplicated for capturing images of the other eye.

Once an image is acquired, it may be pre-processed. Pre-processing may occur either using the computing device 110 of the apparatus, or in the cloud-based network 112. One form of pre-processing that may occur include segmentation. For example, the image of the sclera of the eye 106 may be segmented for further analysis. In some instances, the sclera may be segmented using trained artificial intelligence software. In addition, in some instances, once segmented the sclera images may be reviewed by a person such the trained person 116, described above. Once segmented, the full scleral segmentation can be used for accurate detection of room lighting color and brightness. Blue, red and green pixels selected from the segmented sclera are used to detect room lighting color and brightness.

In some instances, once room lighting color and brightness are determined, an algorithm specific to the determined room lighting color and brightness is selected to make a determination about the eye. For example, separate algorithms may exist for 2700K lighting, 3500K lighting, and 5000K lighting. Based upon the determined room lighting color and brightness, either the 2700K lighting algorithm, the 3500K algorithm, or the 5000K lighting algorithm are selected to determine the refractive error of the eye.

In other instances, trained artificial intelligence software makes a determination about the refractive error of the eye using the determined room lighting color and brightness without having separate algorithms based on room lighting.

Returning to FIGS. 1B and 1C, in some instances the processor 104 can further execute computer-readable instructions stored on the memory 108 to detect, from the image of the eye 106 of the subject, ambient light reflected out of the eye 106 of the subject from the retina of the eye 106 of the subject and to make a determination about the eye 106 of the subject based upon the detected reflected ambient light. Generally, the processor 104 of the apparatus 100 executing computer-readable instructions stored in the memory 108 that cause the processor 104 to make a determination about the eye 106 of the subject based at least in part on an aspect of the reflected ambient light. Such aspects can include, for example, an overall brightness or intensity of the reflected ambient light as determined in a plurality of pixels of the image acquired by the image capture device 102. The aspects can also include one or more colors of the reflected ambient light also as determined from the plurality of pixels of the image acquired by the image capture device 102. For example, the processor 104 executing computer-readable instructions stored in the memory 108 can cause the processor 104 to make a determination about the eye 106 based at least in part on the overall brightness or intensity of the red, green and blue pixels that comprise the reflected ambient light as determined from the image acquired by the image capture device. Overall brightness can be determined, as a non-limiting example, using methods and software developed by Allan Hanbury (see, for example, “A 3D-Polar Coordinate Colour Representation Well Adapted to Image Analysis,” Hanbury, Allan; Vienna University of Technology, Vienna, Austria, 2003), which is fully incorporated herein by reference and made a part hereof. The processor 104 also uses the relative intensity of red, green or blue found in the plurality of pixels of the image acquired by the image capture device 102 to make the determination about the eye 106. For example, using at least in part on an aspect of the reflected ambient light as determined from an image of the eye 106 as captured by the image capture device 102, the processor 104 executing computer-readable instructions stored in the memory 108 can make determinations about the eye 106 comprising a refractive error for the eye 106 of the subject. In other words, using at least in part an overall brightness or intensity of the reflected ambient light as determined in a plurality of the pixels of the image acquired by the image capture device 102 and the relative intensity of one or more colors of the reflected ambient light also as determined from the plurality of pixels of the image acquired by the image capture device 102, the processor 104 executing computer-readable instructions stored in the memory 108 can make determinations about the eye 106 including a refractive error for the eye 106 of the subject.

In other instances, the image of the eye (either before or after pre-processing) can be passed wirelessly from the apparatus 100 to the cloud-based network 112 where one or more processors and one or more memories associated with the cloud-based network 112 can make a determination about the eye 106 based at least in part on the overall brightness or intensity of the red, green and blue pixels that comprise the reflected ambient light as determined from the image acquired by the image capture device. Using at least in part an overall brightness or intensity of the reflected ambient light as determined in a plurality of the pixels of the image acquired by the image capture device 102 and the relative intensity of one or more colors of the reflected ambient light also as determined from the plurality of pixels of the image acquired by the image capture device 102, the cloud-based network processor executing computer-readable instructions stored in the cloud-based network memory can make determinations about the eye 106 including a refractive error for the eye 106 of the subject.

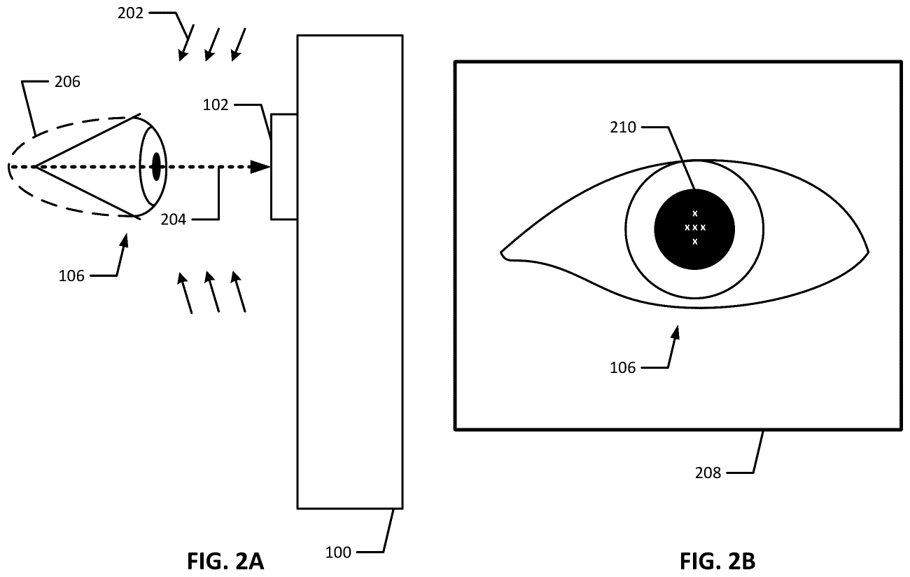

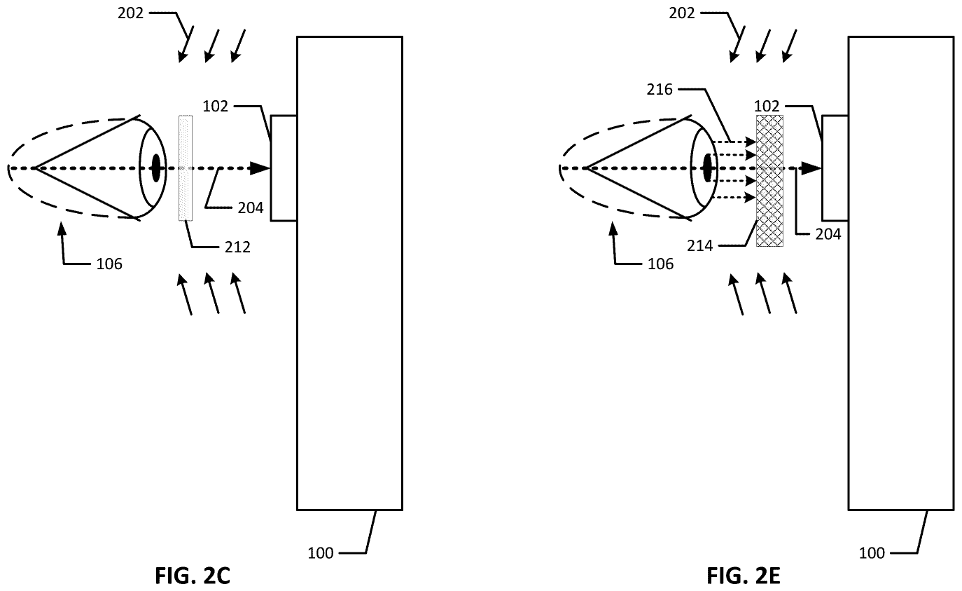

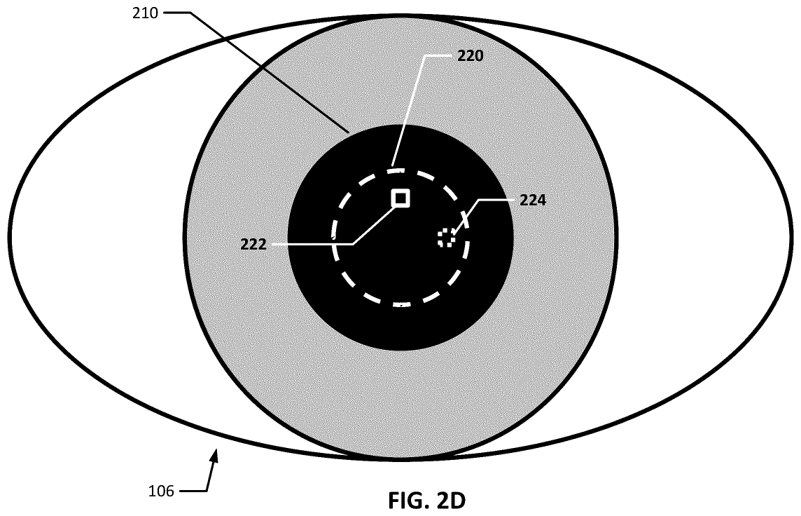

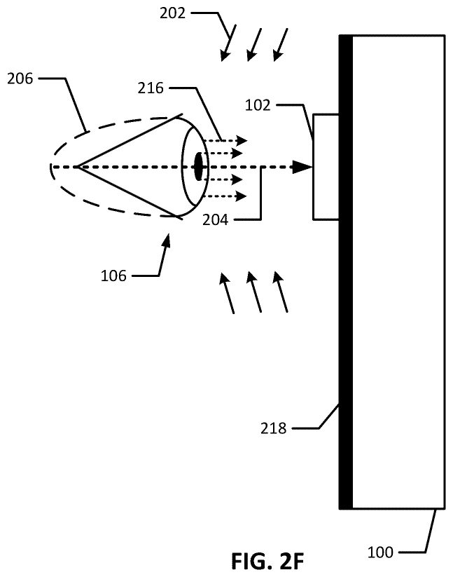

As shown in FIG. 2A, once aligned with the eye 206, the image capture device 102 of the apparatus 100 captures an image (FIG. 2B) 208 of the eye 106. In some instance, the processor 104 of the apparatus 100 can execute computer-readable instructions stored in the memory 108 that cause the processor 104 to pre-process the image 208 of the eye 106. For example, the processor 104 may segment the image 208 of the sclera. The image of the eye 106 is then transmitted, wirelessly, to the cloud-based network 112. A processor associated with the cloud-based network 112 then executes software stored in a memory associated with the cloud-based network 112 to detect, from the image 208 of the eye 206, ambient light 202 reflected 204 out of an eye 106 of the subject from the retina 206 of the eye 106 of the subject and determine the overall intensity of the plurality of pixels (example pixels are shown in FIG. 2B as white “x” the pupil 210 of the image 208 of the eye) within the pupil 210 or a portion of the pupil 210; determine an intensity of a first color from a the plurality of pixels located within the pupil 210 or at least a portion of a pupil 210 of the eye of the subject captured in the image 208; determine an intensity of a second color from the plurality of pixels located within the pupil 201 or at least a portion of the pupil 210 of the eye of the subject captured in the image 208; and calculate refractive error or glasses prescription based on regression analysis. The regression analysis includes at least one of the following elements (1) the overall intensity or brightness of the pixels within pupil 210 or a portion of the pupil 210; and (2) the relative intensity of a first color from a first one or more pixels located within at least a portion of a pupil 210 of the eye of the subject captured in image 208 as compared to a second color from a second one or more pixels located within the at least a portion of the pupil 210 of the eye of the subject captured in image 208. Optionally, the regression analysis can also include (3) the color of the iris of the subject captures in image 208; and (4) the overall intensity of the ambient lighting at the time the image is captured with the image capturing device 100. For example, when the intensity of the first color is brighter relative to the intensity of the second color and the overall intensity is relatively brighter, the determination about the eye of the subject based upon the reflected ambient light can comprise a positive value or hyperopia. Alternatively, when the intensity of the first color is dimmer relative to the intensity of the second color and the overall intensity is relatively dimmer, the determination about the eye of the subject based upon the reflected ambient light can comprise a negative value or myopia.

For example, the first color can comprise any one or any combination of red, green, and blue and the second color can comprise any one or combination of red, green, and blue that is not used as the first color.

By performing the steps described above, the processor associated with the cloud-based network 112 can execute computer-readable instructions stored in the memory associated with the cloud-based network 112 that cause the processor to make an autorefraction or a photorefraction measurement. Such a measurement can be reviewed, confirmed and/or adjusted by a trained person 116. For example, as shown in FIG. 2C, the apparatus 100 can capture, using the image capture device 102, an image 208 of the eye 106 of the subject using only ambient lighting 202 conditions through a spectacle lens or a contact lens (both shown as 212 in FIG. 2C) while the subject is wearing the spectacle lens or the contact lens 212 over the eye 106. The image capturing device 102 of the apparatus 100 then captures a second image using only ambient lighting 202 conditions while the subject is not wearing the spectacle lens or the contact lens 212 over the eye (see, for example, FIG. 2A) and in some instances the processor 104 executes computer-readable instructions stored in the memory 108 that cause the processor 104 to pre-process one or both of the first image and the second image. In some instances, the first image and the second image are transmitted (either before or after pre-processing) to the cloud-based network 112 and a processor associated with the cloud-based network executes computer-executable instructions to compare the first image to the second image and make a determination about the eye of the subject based upon the reflected 204 ambient light is based on the comparison and comprises an estimated prescription for the spectacle lens or the contact lens 212. In some instances, the prescription can be reviewed, confirmed and/or adjusted by a trained person 116.

Referring now to FIG. 2D, in yet another aspect, the processor associated with the cloud-based network 112 can execute computer-readable instructions stored in the memory associated with the cloud-based network 112 that cause the processor to make a first determination about the eye 106 of the subject based upon the reflected ambient light from a first plurality of pixels 220 located within the at least a portion of the pupil 210 of the eye 106 of the subject captured in the image 208; make a second determination from a second plurality of pixels 222 located within the at least a portion of the pupil 210 of the eye 106 of the subject captured in the image 208, wherein the second plurality of pixels 222 are a subset of the first plurality of pixels 210; make a third determination from a third plurality of pixels 224 located within the at least a portion of the pupil 210 of the eye 106 of the subject captured in the image 208, wherein the third plurality of pixels 224 are a subset of the first plurality of pixels 210 and are separate from the second plurality of pixels 222; and compare the first determination, the second determination and the third determination to make the determination about the eye 106 of the subject based upon the reflected ambient light. For example, comparing the first determination, the second determination and the third determination to make the determination about the eye 106 of the subject based upon the reflected ambient light can comprise one or more of determining a standard deviation of the first determination to the second determination, a standard deviation of the first determination to the second determination, or a standard deviation of the second determination to the third determination, wherein the determined standard deviation indicates the determination about the eye 106 of the subject based upon the reflected ambient light. The determination made about the eye 106 of the subject based upon the reflected ambient light can be the presence or absence of astigmatism. The amount of astigmatism, once detected, can be determined by comparing the overall intensity and the relative intensity of the first color or the relative intensity of the second color of various regions of the pupil. For example, measuring one or more of hyperopia or myopia at the various regions of the pupil using the apparatus 100, as described herein, can be used to determine the amount of astigmatism present in the eye 106.

Consider the following example, again referring to FIG. 2D. If a determination of the eyes using the methods and apparatus described herein on the central region of the pupil (entire white dashed circle) 220 for someone with myopia (Ex: −2.00) and no astigmatism, a value of −2.00 would also be obtained in the sub-regions at 90 degrees (solid square) 222 and 0 degrees (dashed square) 224. If someone has astigmatism, a refractive error of −2.00 may be obtained if the whole pupil central region of the pupil (entire white dashed circle) 210 is analyzed using the methods and apparatus described herein, but if the sub-region at 90 degrees (solid square) 222 is analyzed and determined to have a refractive error of −1.00 and the sub-region at 0 degrees (dashed square) 224 is analyzed and determined to have a refractive error of −3.00, the standard deviation would be higher in the case of astigmatism where the sub-regions 222, 224 would be −1.00 and −3.00, respectively. Thus, a prescription for corrective lenses also needs to be −1.00 and −3.00 in those two sub-regions 222, 224, rather than an overall −2.00 for the central pupil region 220. These numbers are also just examples. They could be positive, negative, or both (one of each). Also, many sub-regions can be evaluated to make a determination about the eye. In this example the two sub-regions are at 90 degrees and 0 degrees, but they could be at any location throughout the pupil 210.

As described herein, the apparatus 100 or the image capture device 102 can be used to manage non-relevant reflections from a cornea and a lens of the eye 106 of the subject while capturing the image 208. Such non-relevant reflections can affect the determination about the eye of the subject based upon the reflected ambient light. Managing the non-relevant reflections can include, for example and as shown in FIG. 2E, the use of a polarizing filter 214, wherein non-relevant reflections 216 from the eye 106 are managed while capturing the image 208 by placing the polarizing filter 214 over a lens of the image capture device 102 or between the image capture device 102 and the eye 106 of the subject when capturing the image 208.

In yet another aspect, as shown in FIG. 2F, the apparatus 100 can further comprise a surface 218, wherein non-relevant reflections 216 from the eye 106 are managed while capturing the image 208 comprise the surface 218 absorbing light or preventing the non-relevant reflections 216 from the eye 106 while capturing the image 208. For example, when acquiring the image 208 the apparatus 100 including the image capture device 102 can be placed close to the eye 106 such that non-relevant reflections 216 are minimized and those that do occur are absorbed or prevented by the surface 218. For example, the apparatus 100 including the image capture device 102 can be placed from approximately 4 to 10 cm away from the eye 106 while capturing the image 208, or the apparatus 100 including the image capture device 102 can be placed from approximately 8 to 9 cm away from the eye 106 while capturing the image 208. The surface 218 can comprise, for example, a surface having a black matte finish to facilitate the absorption of ambient light and prevent of non-relevant reflections. The surface 218 can comprise a portion of the image capture device 102 or the apparatus 100, including a case that may house at least a portion of the image capture device 102 or the apparatus 100. For example, the image capture device 102 may comprise at least a portion of a smart phone or other mobile computing device having a camera and the surface 218 can be at least a portion of a case that houses the smart phone or other mobile computing device having a camera.

This disclosure contemplates apparatus that can be used make determinations about the eye 106 in eyes that have smaller than average pupil diameters such as, for example, approximately 2 mm or less. This is currently a challenge for many photorefractors that require assessing the slope of the reflected light over a wide pupil diameter, making it is less useful in more brightly lit rooms or in older patients who have smaller pupils. Further, embodiments of the apparatus described herein can monitor the reflected light in just the center region of the pupil in this measurement allowing accurate measurement of the smaller pupil.

Further, embodiments of the apparatus described herein can monitor the reflected light in a natural pupil or an artificial pupil. An artificial, or second pupil can be optically created for an eye by combining lenses and apertures, without placing anything inside the eye. Vision scientists regularly create what is called a Maxwellian View during experiments where they want to give all subjects the same pupil size by creating an artificial pupil. An artificial pupil could be optically created or physically created by placing an aperture in front of the eye.

Alternatively or optionally, the apparatus 100 as described herein can be used to make a determination of the subject's left eye or right eye. Similarly, it can be used to make a determination of the subject's left eye and right eye.

Though not shown in FIG. 1C, the apparatus 100 can optionally include a light meter or any other mechanism for measure ambient lighting levels. The light meter can detect an intensity for the ambient light conditions and provide an indication if the ambient light conditions are too low for the apparatus 100 to capture an image of the eye of the subject based upon the reflected ambient light. In another aspect, the light meter can measure ambient lighting conditions and such measurement can be used to adjust the image or the calculation of refractive error using regression analysis accordingly.

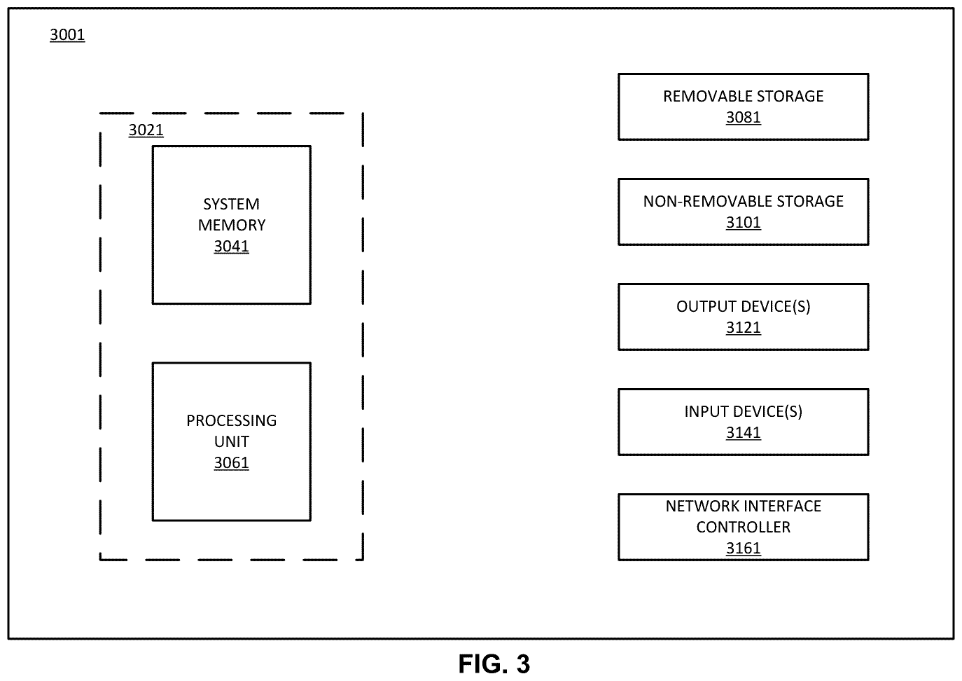

When the logical operations described herein are implemented in software, the process may execute on any type of computing architecture or platform. Such a computing device 3001 as shown in FIG. 3 can be the same as computing device 110, described above, or used alternatively for computing device 110. Furthermore, such a computing device 3001 as shown in FIG. 3 may comprise all or part of cloud-based network 112. For example, referring to FIG. 3, an example computing device 3001 upon which embodiments of the invention may be implemented is illustrated. The computing device 3001 can optionally be a mobile computing device such as a laptop computer, a tablet computer, a mobile phone and the like. The computing device 3001 may include a bus or other communication mechanism for communicating information among various components of the computing device 3001. In its most basic configuration, computing device 3001 typically includes at least one processing unit 3061 and system memory 3041. Depending on the exact configuration and type of computing device, system memory 3041 may be volatile (such as random access memory (RAM)), non-volatile (such as read-only memory (ROM), flash memory, etc.), or some combination of the two. This most basic configuration is illustrated in FIG. 3 by dashed line 3021. The processing unit 3061 may be a standard programmable processor that performs arithmetic and logic operations necessary for operation of the computing device 3001.

Computing device 3001 may have additional features/functionality. For example, computing device 3001 may include additional storage such as removable storage 3081 and non-removable storage 3101 including, but not limited to, magnetic or optical disks or tapes. Computing device 3001 may also contain network connection(s) 3161 that allow the device to communicate with other devices. Computing device 3001 may also have input device(s) 3141 such as a keyboard, mouse, touch screen, etc. Output device(s) 3121 such as a display, speakers, printer, etc. may also be included. The additional devices may be connected to the bus in order to facilitate communication of data among the components of the computing device 3001. All these devices are well known in the art and need not be discussed at length here.

The processing unit 3061 may be configured to execute program code encoded in tangible, computer-readable media. Computer-readable media refers to any media that is capable of providing data that causes the computing device 3001 (i.e., a machine) to operate in a particular fashion. Various computer-readable media may be utilized to provide instructions to the processing unit 3061 for execution. Common forms of computer-readable media include, for example, magnetic media, optical media, physical media, memory chips or cartridges, or any other non-transitory medium from which a computer can read. Example computer-readable media may include, but is not limited to, volatile media, non-volatile media and transmission media. Volatile and non-volatile media may be implemented in any method or technology for storage of information such as computer readable instructions, data structures, program modules or other data and common forms are discussed in detail below. Transmission media may include coaxial cables, copper wires and/or fiber optic cables, as well as acoustic or light waves, such as those generated during radio-wave and infra-red data communication. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 3061 may execute program code stored in the system memory 3041. For example, the bus may carry data to the system memory 3041, from which the processing unit 3061 receives and executes instructions. The data received by the system memory 3041 may optionally be stored on the removable storage 3081 or the non-removable storage 3101 before or after execution by the processing unit 3061.

Computing device 3001 typically includes a variety of computer-readable media. Computer-readable media can be any available media that can be accessed by device 3001 and includes both volatile and non-volatile media, removable and non-removable media. Computer storage media include volatile and non-volatile, and removable and non-removable media implemented in any method or technology for storage of information such as computer readable instructions, data structures, program modules or other data. System memory 3041, removable storage 3081, and non-removable storage 3101 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 3001. Any such computer storage media may be part of computing device 3001.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

The techniques for making a determination about an eye in ambient lighting conditions described herein can optionally be at least partially implemented with a mobile computing device, such as a laptop computer, tablet computer or mobile phone. Accordingly, the mobile computing device is extremely small compared to conventional devices and is very portable, which allows the mobile computing device to be used wherever needed. Many conventional devices have a chin rest that requires the subjects to only look straight ahead during this testing. Unlike conventional devices, the mobile computing device can be placed in any position relative to the subject's head where the eyes can still be viewed and measurements can be made.

It should be appreciated that the logical operations described herein with respect to the various figures may be implemented (1) as a sequence of computer implemented acts or program modules (i.e., software) running on a computing device, (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

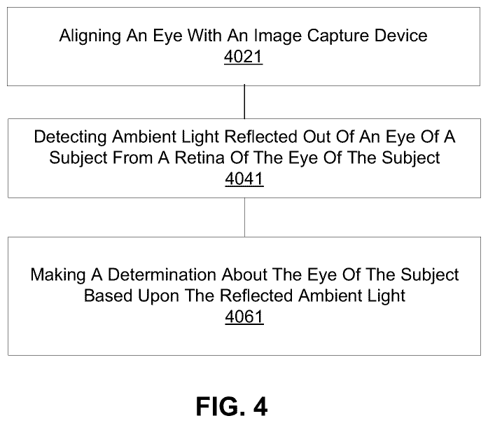

FIG. 4 illustrates an example method for making a determination about an eye of a subject based upon ambient light reflected out of the eye. The method comprises step 4021, aligning the eye with an image capture device, as described herein. At step 4041, detecting, using a computing device, ambient light reflected out of an eye of a subject from a retina of the eye of the subject; and step 4061, making a determination about the eye of the subject based upon the reflected ambient light. In some instances, a color temperature of the ambient light may be detected and used to make the determination about the eye of the subject based upon the reflected ambient light, where the reflected ambient light is adjusted based on the determined color temperature of the ambient lighting.

Making the determination about the eye of the subject based upon the reflected ambient light comprises making a determination based at least in part on an aspect of the reflected ambient light. The aspects can include making a determination based at least in part on an overall brightness (luminescence) of an image of the eye and the intensity of one or more colors of the reflected ambient light. Consider one non-limiting example where the determination about the eye of the subject comprises refractive error and the refractive error is determined by a formula developed through regression analysis. The example formula considers overall brightness (“LuminancePupil”) of the pupil from the image capture using only ambient light and the intensity of blue from one or more pixels from the pupil in the image (“BluePixel”), the intensity of red in one or more pixels from the pupil in the image (“RedPixel”), and the intensity of green in one or more pixels from the pupil in the image (“GreenPixel”) while controlling for ambient light levels (“LuminanceAmbient”). The example formula comprises: Refractive Error=−36.47+(−638.37*RedPixel)+(−1807.2*GreenPixel)+(−333.64*BluePixel)+(2156.5*LuminancePupil)+(183.0*LuminanceAmbient)m+(890.2*GreenPixel*LuminanceAmbient)+(−4895.0*RedPixel*RedPixel)+(−8457.1*GreenPixel*GreenPixel)+(−1711.4*BluePixel*BluePixel)+(1592.8*LuminancePupil*LuminancePupil)+(−178.7*LuminanceAmbient*LuminanceAmbient), and has an R² of approximately 0.78 for fitting the measurement to the intended refractive error of the eye. It is to be appreciated that this is only one example of a formula for making a determination about the eye and other formulas are contemplated within the scope of this disclosure.

Referring back to the method described in FIG. 4, detecting ambient light reflected out of an eye of a subject from a retina of the eye of the subject can further comprise capturing, using an image capture device, an image of the eye of a subject, wherein the image is captured using only ambient lighting conditions and wherein non-relevant reflections from the eye of the subject are managed while capturing the image; determining, using the computing device, an overall intensity of light from a plurality of pixels located within at least a portion of a pupil captured in the image; determining, using the computing device, a first intensity of a first color from the plurality of pixels located within at least a portion of a pupil of the eye of the subject captured in the image; determining, using the computing device, a second intensity of a second color from the plurality of pixels located within the at least a portion of the pupil of the eye of the subject captured in the image; and comparing, by the computing device, a relative intensity of the first color and a relative intensity of the second color, wherein the comparison and the overall intensity are used to make the determination about the eye of the subject based upon the reflected ambient light. For example, when the intensity of the first color is brighter relative to the intensity of the second color and an overall intensity is relatively brighter, the determination about the eye of the subject based upon the reflected ambient light comprises a positive value or hyperopia. Conversely, when the intensity of the first color is dimmer relative to the intensity of the second color and an overall pixel intensity is relatively dimmer, the determination about the eye of the subject based upon the reflected ambient light comprises a negative value or myopia. The first color can comprise any one or any combination of red, green and blue and the second color can comprise any one or any combination of red, green and blue.

In the method of FIG. 4, the determination about the eye of the subject based upon the reflected ambient light can alternatively or optionally comprise an autorefraction or a photorefraction measurement. Capturing, using the image capture device, an image of the eye of the subject can comprise capturing a first image using only ambient lighting conditions with the image capture device through a spectacle lens or a contact lens while the subject is wearing the spectacle lens or the contact lens over the eye and capturing a second image using only ambient lighting conditions with the image capture device while the subject is not wearing the spectacle lens or the contact lens over the eye and the aspects of the reflected ambient light in the first image can be compared to the aspects of the reflected ambient light in the second image and the determination about the eye of the subject based upon the reflected ambient light is based on the comparison and comprises an estimated prescription for the spectacle lens or the contact lens.

The method shown in FIG. 4 can further comprise making a first determination about the eye of the subject based upon the reflected ambient light from a first plurality of pixels located within the portion of the pupil of the eye of the subject captured in the image; making a second determination from a second plurality of pixels located within the portion of the pupil of the eye of the subject captured in the image, wherein the second plurality of pixels are a subset of the first plurality of pixels; making a third determination from a third plurality of pixels located within the portion of the pupil of the eye of the subject captured in the image, wherein the third plurality of pixels are a subset of the first plurality of pixels and are separate from the second plurality of pixels; and comparing the first determination, the second determination and the third determination to make the determination about the eye of the subject based upon the reflected ambient light. Comparing the first determination, the second determination and the third determination to make the determination about the eye of the subject based upon the reflected ambient light can comprise one or more of determining a standard deviation of the first determination to the second determination, a standard deviation of the first determination to the second determination, or a standard deviation of the second determination to the third determination, wherein the determined standard deviation indicates the determination about the eye of the subject based upon the reflected ambient light. For example, the determination about the eye of the subject based upon the reflected ambient light can be the presence or the absence of astigmatism. When the presence of astigmatism is detected, an amount of astigmatism can be determined by comparing the overall intensity and the relative intensity of the first color or the relative intensity of the second color of various regions of the pupil. Such measurements of various regions of the pupil can comprise measuring one or more of hyperopia or myopia at the various regions of the pupil.

As noted above, the method of FIG. 4 can include managing non-relevant reflections from the eye while capturing the image, which can comprise managing reflections from a cornea or a lens of the eye of the subject while capturing the image. For example, a polarizing filter can be placed over a lens of the image capture device or between the image capture device and the eye of the subject. Managing non-relevant reflections from the eye while capturing the image can also comprise blocking light that would lead to reflections from a corneal surface of the eye or a lens of the eye. For example, a surface can be provided that absorbs light or prevents the non-relevant reflections from the eye while capturing the image. In one aspect, the surface can have a black matte finish. In various aspects the surface can comprise a portion of the image capture device or at least a portion of a case that houses the image capture device.

In all instances of the described method of FIG. 4, a color temperature of the ambient light may be detected and used in making determinations about the eye based on the reflected ambient light.

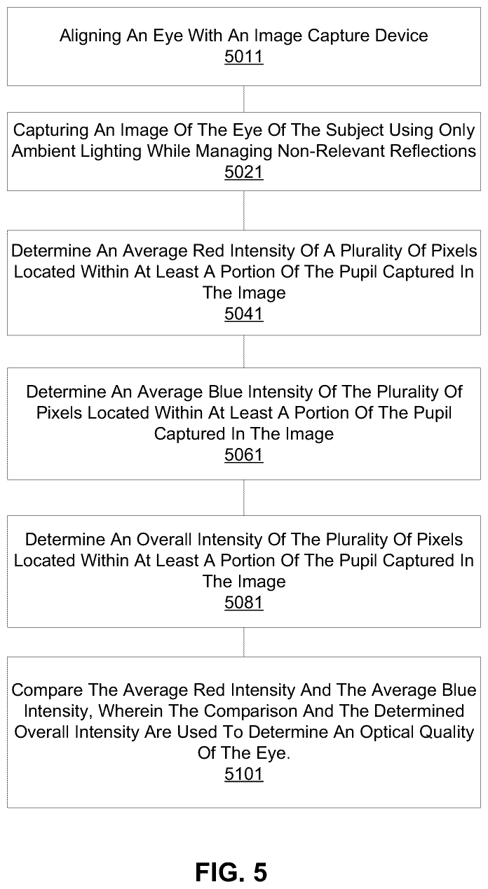

FIG. 5 illustrates an alternate example method for making a determination about an eye of a subject based upon ambient light reflected out of the eye. The method comprises step 5011, aligning the eye with the image capture device. At step 5021, capturing, using the image capture device, an image of an eye of a subject, wherein said image is captured using only ambient lighting conditions and wherein non-relevant reflections from a cornea and a lens of the eye of the subject are managed while capturing the image. At step 5041, an average red intensity can be determined from a plurality of pixels located within at least a portion of a pupil captured in the image. At step 5061, an average blue intensity is determined from the plurality of pixels located within the at least a portion of a pupil captured in the image. At step 5081, an overall intensity is determined of the plurality pixels located within the at least a portion of a pupil captured in the image; and, at step 5101, compare the average red intensity and the average blue intensity, wherein the comparison and the determined overall intensity are used to determine an optical quality of the eye.

In the method of FIG. 5, the determination about the eye of the subject based upon the reflected ambient light can alternatively or optionally comprise an autorefraction or a photorefraction measurement. Capturing, using the image capture device, an image of the eye of the subject can comprise capturing a first image using only ambient lighting conditions with the image capture device through a spectacle lens or a contact lens while the subject is wearing the spectacle lens or the contact lens over the eye and capturing a second image using only ambient lighting conditions with the image capture device while the subject is not wearing the spectacle lens or the contact lens over the eye and the aspects of the reflected ambient light in the first image can be compared to the aspects of the reflected ambient light in the second image and the determination about the eye of the subject based upon the reflected ambient is based on the comparison and comprises an estimated prescription for the spectacle lens or the contact lens.

The method shown in FIG. 5 can further comprise determining a presence or an absence of astigmatism. If the presence of astigmatism is indicated, an amount of astigmatism can be determined by comparing optical quality measurements of various regions of the pupil. Such optical quality measurements of various regions of the pupil can comprise measuring one or more of hyperopia or myopia at the various regions of the pupil.

As noted above, the method of FIG. 5 can include managing non-relevant reflections from the eye while capturing the image, which can comprise managing reflections from a cornea or a lens of the eye of the subject while capturing the image. For example, a polarizing filter can be placed over a lens of the image capture device or between the image capture device and the eye of the subject. Managing non-relevant reflections from the eye while capturing the image can also comprise blocking light that would lead to reflections from a corneal surface of the eye or a lens of the eye. For example, a surface can be provided that absorbs light or prevents the non-relevant reflections from the eye while capturing the image. In one aspect, the surface can have a black matte finish. In various aspects the surface can comprise a portion of the image capture device or at least a portion of a case that houses the image capture device.

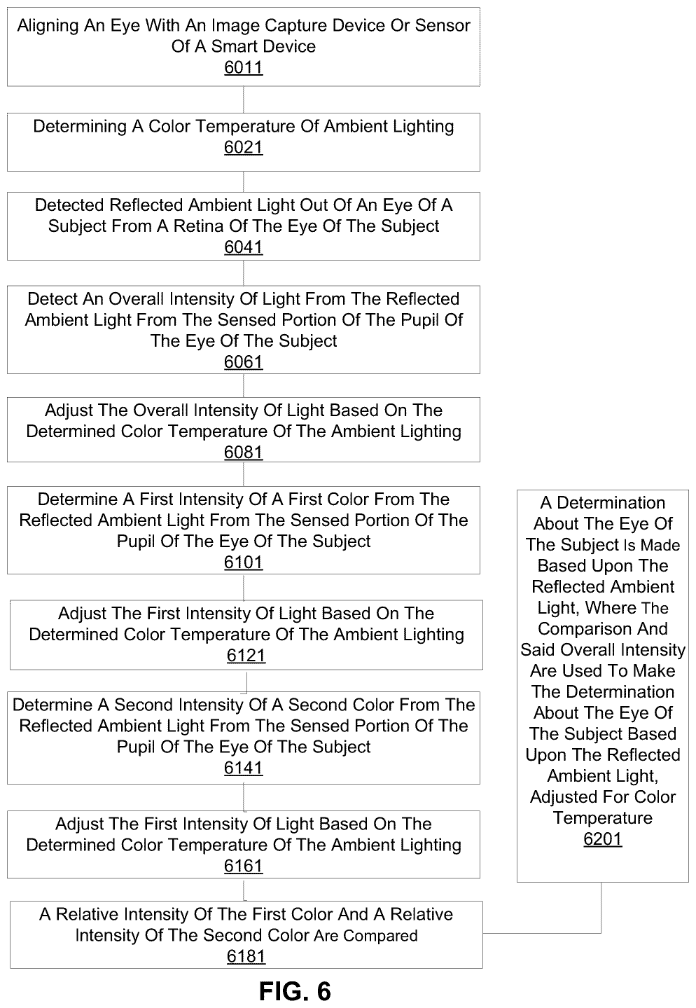

FIG. 6 is a flowchart for a method of making a determination about an eye of a subject based upon ambient light reflected out of the eye. The method comprises 6011, aligning an image capture device or sensor of a smart device with an eye. Step 6021, determining, using a computing device, a color temperature of ambient lighting. In some instances, determining the color temperature of ambient lighting comprises determining, by the computing device, the color temperature of the ambient lighting using the sclera and/or pupil of the eye of the subject, wherein reflected light of the sclera and/or pupil of the eye is sensed by the sensor. In some instances, determining the color temperature of the ambient lighting using the sclera and/or pupil of the eye of the subject comprises using reflected light from the sclera and/or pupil of the eye to sense the color temperature of the ambient lighting. In some instances, determining the color temperature of the ambient lighting using the sclera and/or pupil of the eye of the subject comprises acquiring, in real-time, reflected light from the sclera and/or pupil of the eye that are used by the computing device to sense the color temperature of the ambient lighting. In some instances, the color temperature of the ambient lighting using the sclera and/or pupil of the eye of the subject comprises determining, by the computing device, a hue and/or luminance of the sclera of the eye of the subject and the computing device using the hue and/or luminescence to determine the color temperature of the ambient lighting. In some instances, determining the color temperature of ambient lighting comprises determining, by the computing device, the color temperature of the ambient lighting using an external white balance card wherein reflected light from the white balance card is sensed by the sensor.

At 6041, reflected ambient light out of an eye of a subject from a retina of the eye of the subject is detected. In one aspect the detecting comprises sensing, using a sensor, at least a portion of the eye of the subject, wherein the sensing is performed using only ambient lighting conditions and wherein non-relevant reflections from the eye of the subject are managed while sensing the portion of the eye, and wherein the sensed portion of the eye comprises at least a portion of a pupil of the eye of the subject. In some instances, sensing, using the sensor, the portion of the eye of the subject comprises sensing at a first time through a spectacle lens or a contact lens while the subject is wearing the spectacle lens or the contact lens over the eye and sensing at a second time while the subject is not wearing the spectacle lens or the contact lens over the eye and the first sensing information is compared to the second sensing information and the determination about the eye of the subject based upon the reflected ambient light is based on the comparison and comprises an estimated prescription for the spectacle lens or the contact lens. In some instances, managing non-relevant reflections from the eye while capturing the image comprises managing reflections from a cornea or a lens of the eye of the subject while sensing the eye. In other instances, managing non-relevant reflections from the eye while sensing the eye comprises placing a polarizing filter over a lens of the sensor or between the sensor and the eye of the subject, or wherein managing non-relevant reflections from the eye while sensing the eye comprises blocking light that would lead to reflections from a corneal surface of the eye or a lens of the eye, or wherein managing non-relevant reflections from the eye while sensing the eye comprises providing a surface that absorbs light or prevents the non-relevant reflections from the eye while sensing the eye.

At 6061, an overall intensity of light from the reflected ambient light from the sensed portion of the pupil of the eye of the subject is determined. At 6081, the overall intensity of light is adjusted by the computing device based on the determined color temperature of the ambient lighting. At 6101, a first intensity of a first color from the reflected ambient light from the sensed portion of the pupil of the eye of the subject is determined. At 6121, the first intensity of the first color is adjusted by the computing device based on the determined color temperature of the ambient lighting. At 6141, a second intensity of a second color from the reflected ambient light from the sensed portion of the pupil of the eye of the subject is determined. In some instances, the first color comprises any one or any combination of red, green, and blue and the second color comprises any one or any combination of red, green, and blue. At 6161, the second intensity of the second color is adjusted by the computing device based on the determined color temperature of the ambient lighting. At 6181, a relative intensity of the first color and a relative intensity of the second color are compared, and at 6201 a determination about the eye of the subject is made based upon the reflected ambient light, where the comparison and said overall intensity are used to make the determination about the eye of the subject based upon the reflected ambient light.

In some instances, the first intensity of the first color is brighter relative to the second intensity of the second color and the overall intensity is relatively brighter in luminescence than a myopic eye, and the determination about the eye of the subject based upon the reflected ambient light comprises a positive value or hyperopia.

In some instances, the first intensity of the first color is dimmer relative to the second intensity of the second color and the overall intensity is relatively dimmer in luminescence than a myopic eye, and the determination about the eye of the subject based upon the reflected ambient light comprises a negative value or myopia.

In some instances, the determination about the eye of the subject based upon the reflected ambient light comprises an autorefraction or a photorefraction measurement.

In some instances, the method may further comprise making a first determination about the eye of the subject based upon the reflected ambient light from a first portion of the sensed pupil of the eye; making a second determination from a second portion of the sensed pupil of the eye of the subject, wherein the second portion of the sensed pupil is a subset of the first portion of the sensed pupil of the eye; making a third determination from a third portion of the sensed pupil of the eye of the subject, wherein the third portion of the pupil is a subset of the first portion of the sensed pupil of the eye and is separate from the second sensed portion of the eye; comparing the first determination, the second determination and the third determination to make the determination about the eye of the subject based upon the reflected ambient light. In some instances, comparing the first determination, the second determination and the third determination to make the determination about the eye of the subject based upon the reflected ambient light comprises one or more of determining a standard deviation of the first determination to the second determination, a standard deviation of the first determination to the third determination, or a standard deviation of the second determination to the third determination, wherein the determined standard deviation indicates the determination about the eye of the subject based upon the reflected ambient light. In some instances, the determination about the eye of the subject based upon the reflected ambient light is a presence or an absence of astigmatism. In some instances, the presence of astigmatism is detected and an amount of astigmatism is determined by comparing the overall intensity and the relative intensity of the first color or the relative intensity of the second color of various regions of the pupil.

In other embodiments, the methods, apparatus and systems described herein can be used to determine the presence and/or severity of a cataract within the eye. Clinicians routinely grade cataracts and other ocular media distortions or opacities based on severity. Because cataracts/opacities affect the optical quality of the eye, patients will experience a decline in their vision, and the severity of the cataract/opacity will be correlated with the decline in a patient's visual acuity measurements, or ability to read a letter chart. The accuracy of refractive error measurements of the eye are dependent upon the ability of light to travel through the eye, so cataracts/opacities will also reduce the accuracy of these measurements.

Cataracts are labeled as Nuclear, Cortical, and Subcapsular. While all three affect the optical quality of the eye and clarity of vision, nuclear cataracts cause a distinct change in color to the lens known as brunescence, or a progressive yellowing, browning, and eventually reddening of the crystalline lens, and the brunescence will also change the color of the light reaching the retina. The cortical and subcapsular cataracts are opacities of the lens that may change the color of the lens to some degree. Opacities in the crystalline lens, cornea, and/or vitreous will also distinctly scatter light. When patients have any cataract or other ocular media opacity, reduced visual acuity scores will be the first sign of this problem for the clinician, and then he or she will know that refractive error measurements of the eye, including autorefraction, may have reduce accuracy. Currently-marketed autorefractors or photorefractors do not, however, alert a clinician to this problem. For the methods, apparatus, and systems described herein, as the cataracts/opacities change the color of the light reaching the retina, they will also change the color of the light reflecting back from the retina and therefore the relative ratio of the RGB pixel values that are contained within the pupil in the image. In a given color of room lighting, the combined color RGB pixel values within the pupil of a cataract patient will be relatively more yellow/brown/red or “brunescent” than what would be observed in patients without cataracts and a more severe cataract has will have a relatively greater effect on the color of the pixel values. In the case of opacities that scatter light, the degree of noise, as defined by a relatively higher standard deviation of RGB pixel values within the pupil with the opacity as compared to a pupil without an opacity. This information on color and noise of the RGB pixel values in the pupil can be used to alert the clinician to the presence of cataracts/opacities as well as to grade the severity.

For example, lens opacity qualities in each of the one or more images of the eye of the subject may comprise cortical spoking and an estimated aggregate of cortical spoking in a plurality of images may be determined by comparing the cortical spoking found in each of the images. In some instances, the plurality of images may comprise five, or more, images of the eye of the subject. The presence and/or severity of the cortical spoking is used to determine the presence and/or severity of a cortical cataract.

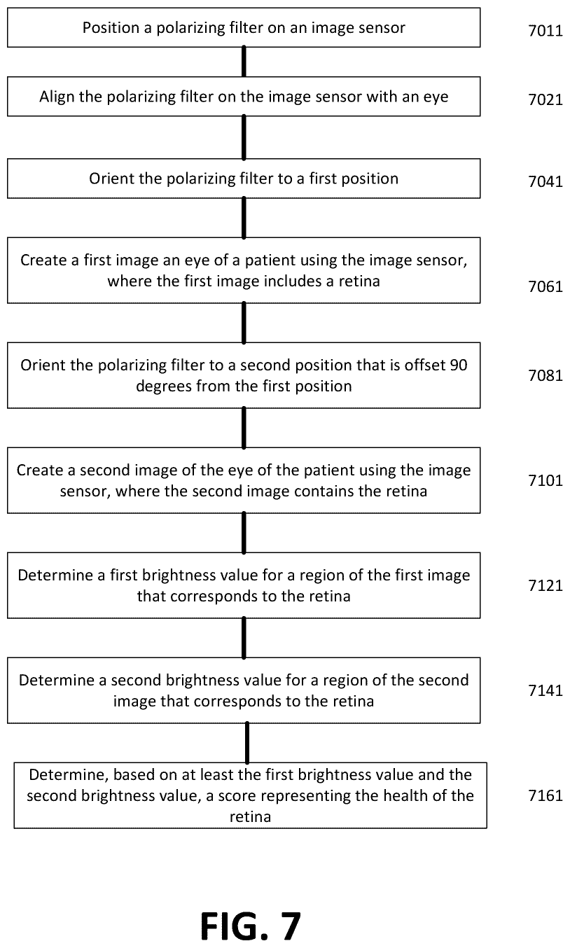

FIG. 7 is a flowchart illustrating an exemplary process for making a determination about an eye. In this instance, the determination comprises measuring retinal nerve fiber layer integrity. At 7011, a polarizing filter us positioned on an image sensor. Generally, the image sensor will comprise a part of a smart device such as a smart phone, table, laptop computer, etc. A typical image sensor is an image acquisition device (e.g., camera) on a smart phone. At 7021, the image sensor and polarizing filter are aligned with the eye using the method described herein. At 7041, the polarizing filter is oriented to a first position. At step 7061, while the polarizing filter is oriented in the first position, an image of the eye is captured. The image includes the image of the pupil of the eye that is produced by the cornea. At step 7081, the polarizing filter is oriented to a second position that is offset by 90 degrees from the first position. For example, the smart phone may be rotated by 90 degrees and re-aligned with the eye. In some instances, the app on the smart phone may instruct a user how to orient the smart phone for capturing a second image and provide visual and/or audible indicators of proper orientation. At step 7101, a second image of the eye is captured. This second image also includes an image of the image of the pupil. At step 7121, a first brightness value for a region of the first image that corresponds to the retina is determined. In some instances, this may occur after the image has been transmitted to the cloud-based network. At step 714, a second brightness value for a region of the second image that corresponds to the image of the image of the pupil is determined. In some instances, this also may occur after the image has been transmitted to the cloud-based network. At step 7161, a score is determined based on at least the first brightness value and the second brightness value, the score representing the health of the retina, as light reflecting off of the retinal nerve fiber layer and back out of the eye has a certain polarization, and would be detected in the image of the image of the pupil. In the case of an unhealthy retinal nerve fiber layer, less light would be reflected and therefore a reduced brightness of the polarized light would be detected within the image of the image of the pupil. This determination may be determined by a processor associated with the cloud-based network and the determination may be reviewed, confirmed or modified by a trained person.

In some instances, automated tropia and phoria measurements can be performed with measurements of the power of the eye obtained with autorefraction. If a subject is looking very far away, the power of the eye that is measured with autorefraction is an estimate of the subject's glasses prescription. If, however, the subject is looking at a near target, an autorefractor can measure how much the subject is focusing to see that near object. The tropia and phoria measurements are always done both while the subject is looking at distance and also while the subject is looking at a near target. It is important that during the distance measurement the eyes are completely relaxed, and that during the near measurement the eyes are focusing accurately. The near tropia and phoria measurements will be different from the distance tropia and phoria measurements only if a subject has an abnormal accommodative convergence accommodation (AC/A) ratio. The AC/A ratio is the amount that they eye turns inwards (e.g., accommodative convergence, AC) for each unit of power for focusing on a near-target (e.g., accommodation, A). Accommodation and accommodative convergence are neurologically linked. If someone with an abnormal AC/A under or over focuses on a near target, the clinician will get a different near phoria or tropia measurement than if the subject is focusing accurately. AC/A can be calculated by having someone look at two or more different targets that require different amounts of accommodation (two different denominators, “A”) and comparing the accommodative convergence (the numerator, “AC”) and calculating the difference between the convergence for the one target and the other target to determine the AC/A. According to the techniques described here, the same camera and light can be used to perform simultaneous tropia/phoria and autorefraction measurements. This allows the clinician to only make the measurement when the subject is accommodating at a certain level, or to adjust the tropia/phoria measurement based on the accommodative effort that was exerted, thus improving the accuracy of the measurement.

In addition, all of these same imaging measurements provide a measurement of each subject's AC/A. Thus, it is possible to determine how much the eye turned inward (e.g., accommodative convergence, AC) from the position of the Purkinje I image for both eyes and how much the subject accommodated (A). Currently, there are no automated measurements of AC/A. Currently, the cover test is performed at multiple distances that require different levels of accommodation, and the ratio is determined from at least two such measurements, or lenses are placed in front of the eye and the clinician assumes that the subject accommodates the same amount as the lenses. A phoria measurement is done with and without the lenses to determine the accommodative convergence (AC).

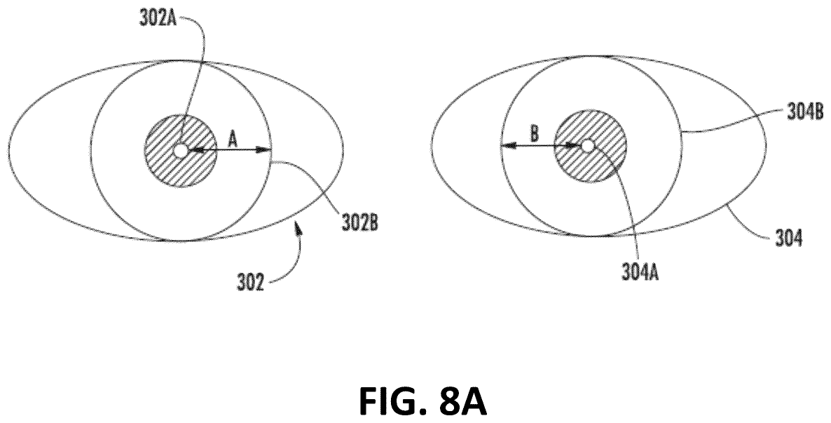

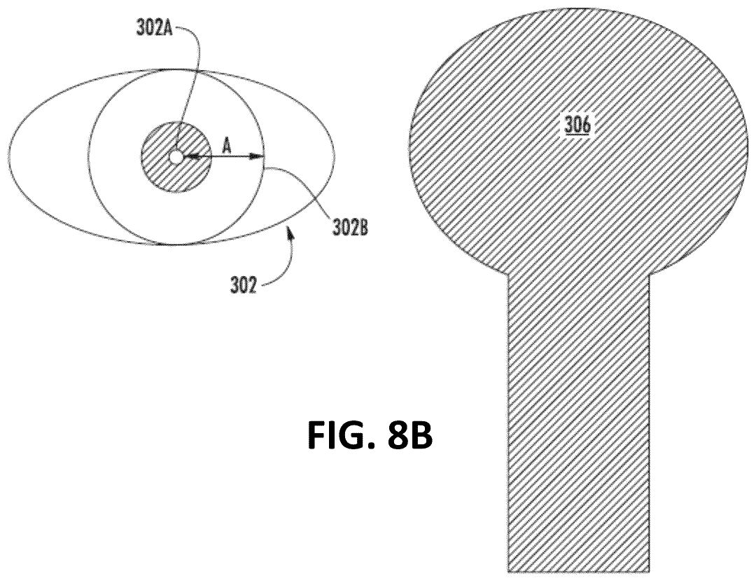

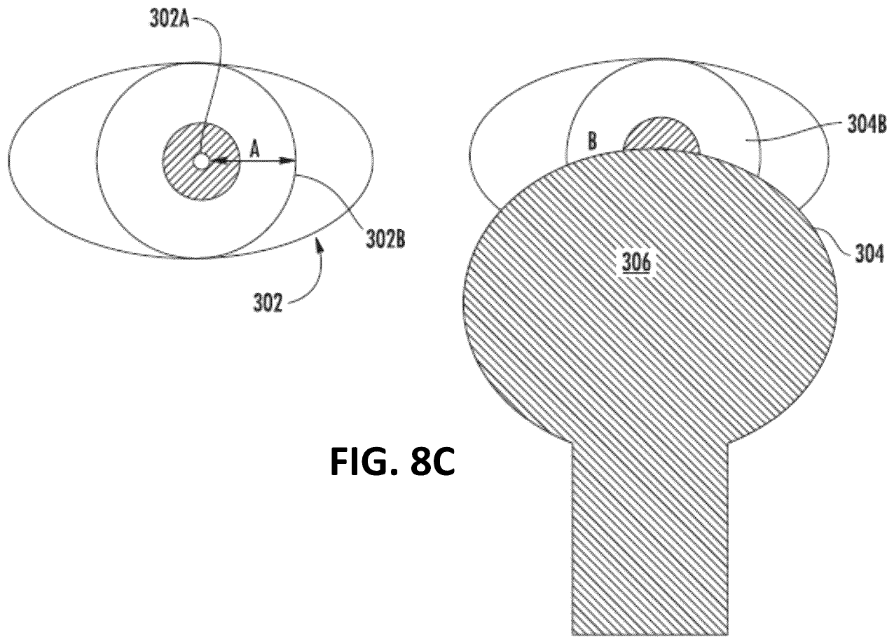

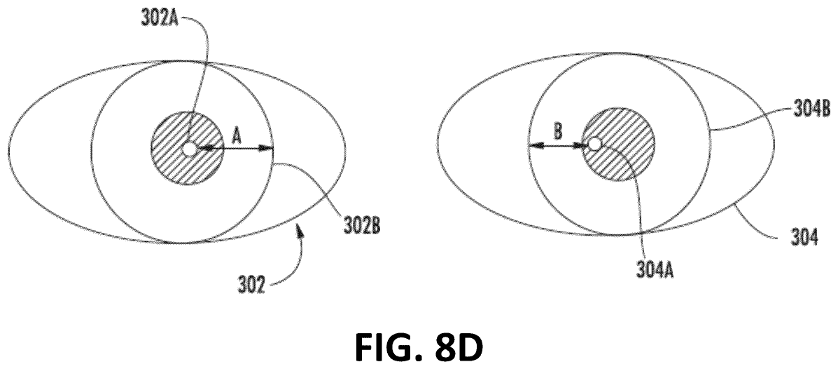

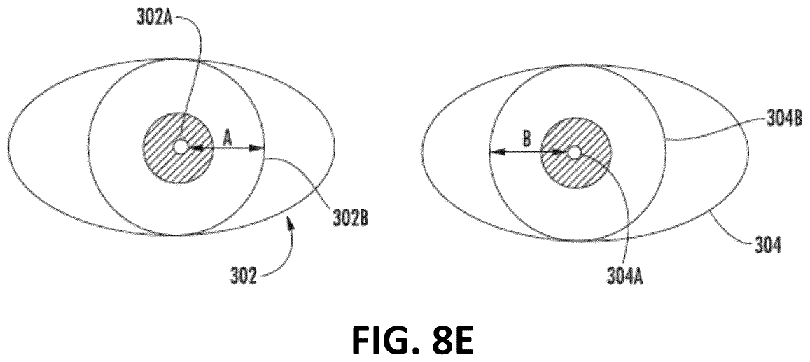

Referring now to FIGS. 8A-8E an example automated test for phoria measurement is shown. In FIG. 8A, the subject's right and left eyes are fixated at the same place. The subject's eyes (e.g., at least one of the subject's eyes) can be illuminated with a light using a light source. Optionally, ambient or available light can be used, wherein no additional light source is required. Optionally, the light can be in a visible or non-visible portion of an electromagnetic spectrum. For example, the light can be infrared or visible light. Although infrared and visible light are provided as examples, this disclosure contemplates the light from other portions of the electromagnetic spectrum can be used.

The subject's eye or eyes can be aligned, either concurrently or serially, with an image capture device of an apparatus such as apparatus 100. In some instances, apparatus 100 can further include one or more light sources. An image of the subject's eyes can be captured using the image capturing device, for example. As shown in FIG. 8A, the image can include a reflection of the light from the subject's eyes or another landmark feature (blood vessel, visible portion of the iris, iris feature, center of the pupil, center of the visible iris diameter, etc.). For example, a reflection of the light 302A from the subject's right eye 302 and a reflection of light 304A from the subject's left eye 304 are shown. Optionally, the image can include a reflection of the light from at least one of an outer or inner surface of a cornea (e.g., a first or second Purkinje image, respectively) of the subject's eyes. Alternatively or additionally, the image can include a reflection of the light from at least one of an outer (anterior) or inner (posterior) surface of a lens (e.g., a third or fourth Purkinje image, respectively) of the subject's eyes. In other words, the image can be a first, second, third or fourth Purkinje image. Although the first through fourth Purkinje images are provided as examples, this disclosure contemplates that the image can include a reflection of the light from any surface of a subject's eye. Further, this disclosure contemplates that any other feature of the eye (blood vessel, visible portion of the iris, iris feature, center of the pupil, center of the visible iris diameter, etc.) can be used to track its position or movement, thus not requiring a reflection.

In FIG. 8A, distance “A” is the distance between the reflection of the light 302A from the subject's right eye 302 and a visible portion of an iris 302B of the subject's right eye 302, and distance “B” is the distance between the reflection of light 304A from the subject's left eye 304 and a visible portion of an iris 304B of the subject's left eye 304. Because distance “A” equals distance “B,” no tropia is present. To determine if a phoria is present, one of the subject's eyes can be sequentially covered and uncovered. Optionally, a sequence of images can be captured after aligning the image capture device with the eye and uncovering one of the subject's eyes. As described below, the reflection of the light within at least one of the subject's eyes in one of the sequence of images can be compared to a position of the reflection of the light within the at least one of the subject's eyes in another of the sequence of images to determine any movement after the subject's eye is uncovered and phoria or tropia magnitude and direction can be calculated from the movement.

In FIG. 8B, the subject's left eye 304 is covered with a cover 306. In FIG. 8C, the subject's left eye 304 is partially uncovered. As described above, images can be captured with sequentially covering and uncovering the subject's left eye 304. In FIG. 8D, the subject's left eye 304 is completely uncovered. Similar to above, an image of the subject's eyes can be captured using the image capturing device when the subject's left eye 304 is completely uncovered. As shown in FIG. 8D, the image can include a reflection of the light from the subject's eyes, e.g., a reflection of the light 302A from the subject's right eye 302 and a reflection of light 304A from the subject's left eye 304 are shown. In FIG. 8D, distance “A” is the distance between the reflection of the light 302A from the subject's right eye 302 and a visible portion of an iris 302B of the subject's right eye 302, and distance “B” is the distance between the reflection of light 304A from the subject's left eye 304 and a visible portion of an iris 304B of the subject's left eye 304. Because distance “A” is not equal to distance “B,” a phoria is present. For example, in FIG. 3D because distance “B” is less than distance “A,” an exophoria is present. The phoria measurement can be determined based on the position of the reflection of the light within the subject's eyes in FIG. 8D.

After approximately 1-2 seconds, the subject's left eye 304 (e.g., the eye that was sequentially covered and uncovered), takes up fixation again on the same place as the subject's right eye 302. Thus, as shown in FIG. 8E, distance “A” is the distance between the reflection of the light 302A from the subject's right eye 302 and a visible portion of an iris 302B of the subject's right eye 302, and distance “B” is the distance between the reflection of light 304A from the subject's left eye 304 and a visible portion of an iris 304B of the subject's left eye 304. Because distance “A” equals to distance “B,” no tropia is present.

It should be appreciated that the logical operations described herein with respect to the various figures may be implemented (1) as a sequence of computer implemented acts or program modules (i.e., software) running on a computing device, (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

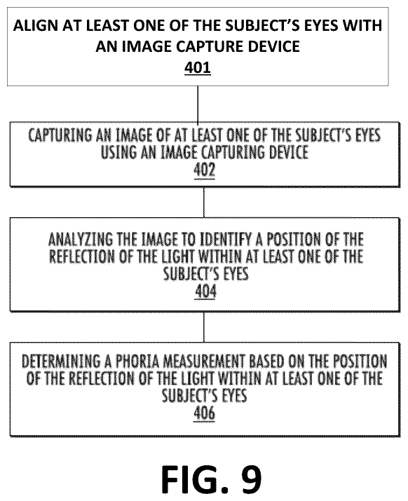

FIG. 9 illustrates an example method for automatically measuring a subject's phoria while the subject fixates on a visual target. This embodiment of a method can include step 401, aligning an eye of a subject with an image capture device, as described herein. Step 402, capturing an image of at least one of the subject's eyes using the image capturing device. The image can include a reflection of the light from at least one of the subject's eyes. The method can also include Step 404, analyzing the image to identify a position of the reflection of the light within at least one of the subject's eyes, and Step 406, determining a phoria measurement based on the position of the reflection of the light within at least one of the subject's eyes.

Optionally, the method can include comparing a position of the reflection of the light within one of the subject's eyes (e.g., a left or right eye) and a position of the reflection of the light within another one the subject's eyes (e.g., the right or left eye). The phoria measurement can be determined based on a result of the comparison.

Optionally, the step of analyzing the image to identify a position of the reflection of the light within at least one of the subject's eyes can include identifying a position of the reflection of the light relative to a landmark of at least one of the subject's eyes.

Optionally, the image can include a reflection of the light from at least one of an outer or inner surface of a cornea (e.g., a first or second Purkinje image, respectively) of at least one of the subject's eyes. Alternatively or additionally, the image can include a reflection of the light from at least one of an outer (anterior) or inner (posterior) surface of a lens (e.g., a third or fourth Purkinje image, respectively) of at least one of the subject's eyes. In other words, the image can be a first, second, third or fourth Purkinje image. Although the first through fourth Purkinje images are provided as examples, this disclosure contemplates that the image can include a reflection of the light from any surface of a subject's eye.

Additionally, the method can optionally include sequentially covering and uncovering at least one of the subject's eyes. Additionally, the image can be captured after uncovering at least one of the subject's eyes. Additionally, the method can optionally include capturing a sequence of images of the subject's eyes after uncovering at least one of the subject's eyes and comparing the reflection of the light within at least one of the subject's eyes in one of the sequence of images to a position of the reflection of the light within the at least one of the subject's eyes in another of the sequence of images to determine any movement after the subject's eye is uncovered.

Alternatively, the method can include covering at least one of the subject's eyes with a filter, wherein the image is captured while at least one of the subject's eyes is covered by the filter. The filter can be opaque to the subject such that the subject cannot see through the filter, but the filter can pass light of a specified wavelength (e.g., infrared light). An example filter is the WRATTEN #89B from EASTMAN KODAK COMPANY of ROCHESTER, NY. It should be understood that the WRATTEN #89B is provided only as an example and that other filters can be used, including filters that pass light with wavelengths other than infrared. Accordingly, the image capturing device can capture the image of at least one of the subject's eyes through the filter. In other words, the alignment measurement can be performed without sequentially covering and uncovering at least one of the subject's eyes.

Optionally, the method can include performing an autorefraction measurement. As used herein, the autorefraction measurement is a measurement a power of a subject's eye by any known technique, including but not limited to, autorefraction or photorefraction. The autorefraction measurement can be taken while the subject is focusing on the visual target, for example. The image can optionally be captured in response to the power of the subject's eye being within a predetermined range. Alternatively or additionally, the method can optionally include adjusting the phoria measurement based on the autorefraction measurement.

Optionally, the method can include calculating an accommodative convergence accommodation ratio based on a position of the reflection of the light within at least one of the subject's eyes and the autorefraction measurement.

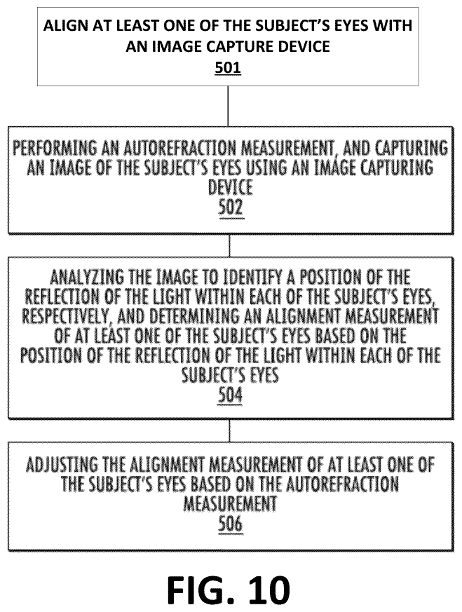

FIG. 10 illustrates a flowchart for an example method for automatically measuring alignment of at least one of a subject's eyes. This embodiment of a method can include step 501, aligning an eye of a subject with an image capture device, as described herein. Step 502, performing an autorefraction measurement using telehealth procedures or when used by a layperson in a vision screening, and capturing an image of the subject's eyes using the image capturing device. As described above, the autorefraction measurement is a measurement a power of a subject's eye by any known technique, including but not limited to, autorefraction or photorefraction. Additionally, the image can include a reflection of the light from each of the subject's eyes. The method can also include Step 504, analyzing the image to identify a position of the reflection of the light within each of the subject's eyes, respectively, and determining an alignment measurement of at least one of the subject's eyes based on the position of the reflection of the light within each of the subject's eyes, respectively.

Optionally, the image is captured in response to the power of at least one of the subject's eyes being within a predetermined range. Alternatively, the method can optionally include Step 506, adjusting the alignment measurement of at least one of the subject's eyes based on the autorefraction measurement. Additionally, the method can optionally include calculating an accommodative convergence accommodation ratio based on a position of the reflection of the light within at least one of the subject's eyes and the autorefraction measurement.

Additionally, the method can optionally include comparing a position of the reflection of the light within one of the subject's eyes (e.g., a left or right eye) and a position of the reflection of the light within another one the subject's eyes (e.g., the right or left eye). The phoria measurement can be determined based on a result of the comparison.

Optionally, the step of analyzing the image to identify a position of the reflection of the light within each of the subject's eyes, respectively, further comprises identifying a position of the reflection of the light relative to a landmark of each of the subject's eyes, respectively.

Optionally, the image can include a reflection of the light from at least one of an outer or inner surface of a cornea (e.g., a first or second Purkinje image, respectively) of at least one of the subject's eyes. Alternatively or additionally, the image can include a reflection of the light from at least one of an outer (anterior) or inner (posterior) surface of a lens (e.g., a third or fourth Purkinje image, respectively) of at least one of the subject's eyes. In other words, the image can be a first, second, third or fourth Purkinje image. Although the first through fourth Purkinje images are provided as examples, this disclosure contemplates that the image can include a reflection of the light from any surface of a subject's eye.

Optionally, the alignment measurement can be a phoria measurement or a tropia measurement.

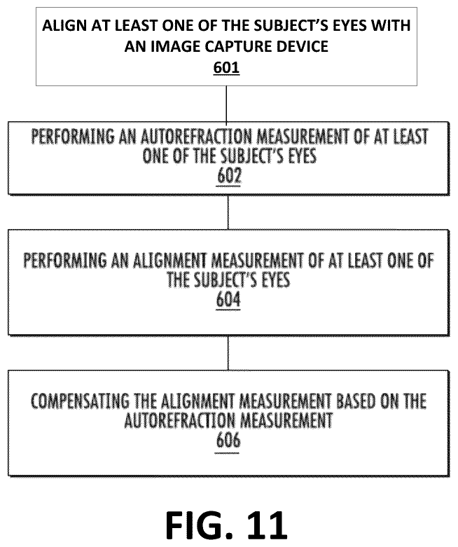

FIG. 11 illustrates a flowchart of another example method for measuring alignment of at least one eye. This embodiment of a method can include step 601, aligning an eye of a subject with an image capture device, as described herein. Step 602, performing an autorefraction measurement using telehealth procedures or when used by a layperson in a vision screening of at least one of a subject's eyes using the captured image, as described herein, Step 604, performing an alignment measurement of at least one of the subject's eyes, and Step 606, compensating the alignment measurement based on the autorefraction measurement.

As described above, the autorefraction measurement is a measurement of the power of a subject's eye by any known technique, including but not limited to, autorefraction or photorefraction. The autorefraction measurement can be taken while the subject is focusing on the visual target, for example. Optionally, the step of compensating the alignment measurement based on the autorefraction measurement includes performing the alignment measurement only when the autorefraction measurement is within a predetermined range. Alternatively, the step of compensating the alignment measurement based on the autorefraction measurement includes adjusting the alignment measurement based on the autorefraction measurement.

Optionally, the alignment measurement can be a phoria measurement or a tropia measurement.

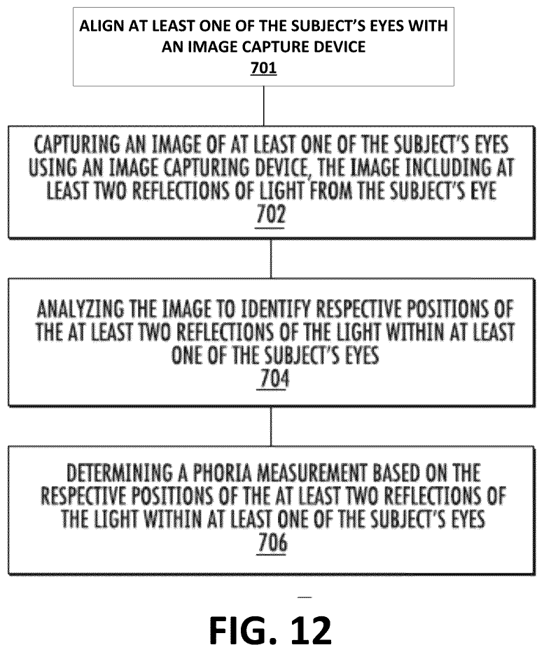

FIG. 12 illustrates a flowchart of another example method for automatically measuring a subject's phoria while the subject fixates on a visual target. This embodiment of a method can include step 701, aligning an eye of a subject with an image capture device, as described herein. Step 702, capturing an image of at least one of the subject's eyes using the image capturing device. The image can include at least two reflections of the light from at least one of the subject's eyes. For example, the image can include at least two reflections of the light from at least two of an outer or inner surface of a cornea (e.g., a first or second Purkinje image, respectively) of at least one of the subject's eyes or an outer (anterior) or inner (posterior) surface of a lens (e.g., a third or fourth Purkinje image, respectively) of at least one of the subject's eyes. This disclosure contemplates that the image can include at least two reflections of the light from any two surfaces of a subject's eyes and should not be limited to the above examples (e.g., the first through fourth Purkinje images). The method can also include Step 704, analyzing the image to identify respective positions of the at least two reflections of the light within at least one of the subject's eyes, and determining a phoria measurement based on the respective positions of the at least two reflections of the light within at least one of the subject's eyes.

Optionally, the method can further include comparing respective positions of the at least two reflections of the light within one of the subject's eyes and respective positions of the at least two reflections of the light within another one the subject's eyes. The phoria measurement can be determined based on a result of the comparison.

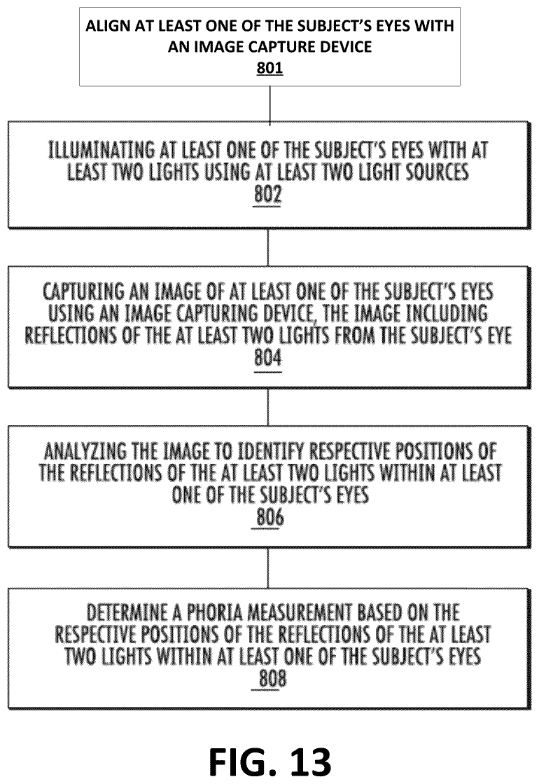

FIG. 13 illustrates a flowchart of yet another example method for automatically measuring a subject's phoria while the subject fixates on a visual target. This embodiment of a method can include step 801, aligning an eye of a subject with an image capture device, as described herein. Step 802, illuminating at least one of the subject's eyes with at least two lights using at least two light sources, and Step 804, capturing an image of at least one of the subject's eyes using an image capturing device. The image can include reflections of the at least two lights from at least one of the subject's eyes. For example, the image can include reflections of the at least two lights from at least one of an outer or inner surface of a cornea (e.g., a first or second Purkinje image, respectively) of at least one of the subject's eyes or an outer (anterior) or inner (posterior) surface of a lens (e.g., a third or fourth Purkinje image, respectively) of at least one of the subject's eyes. This disclosure contemplates that the image can include reflections of the at least two lights from any surface of a subject's eyes and should not be limited to the above examples (e.g., the first through fourth Purkinje images). The method can also include Step 806, analyzing the image to identify respective positions of the reflections of the at least two lights within at least one of the subject's eyes, and Step 808, determining a phoria measurement based on the respective positions of the reflections of the at least two lights within at least one of the subject's eyes.

Optionally, the method can include comparing respective positions of the reflections of the at least two lights within one of the subject's eyes and respective positions of the reflections of the at least two lights within another one the subject's eyes, wherein the phoria measurement is determined based on a result of the comparison.

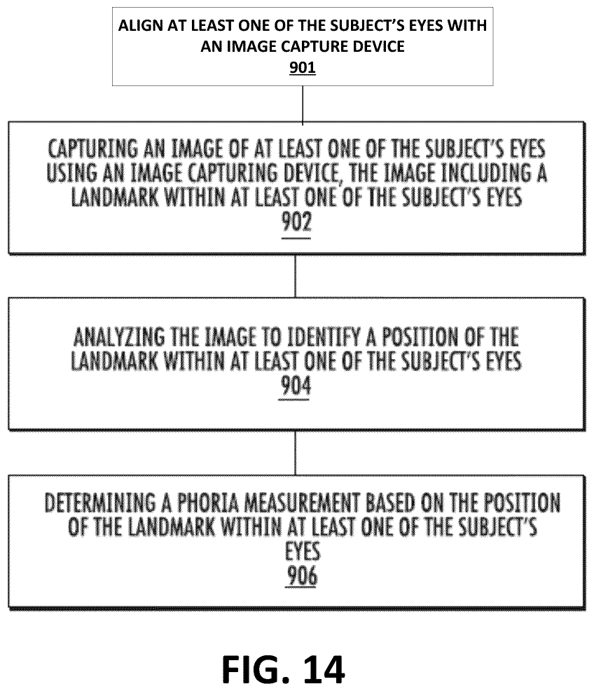

FIG. 14 illustrates a flowchart of another example method for automatically measuring a subject's phoria while the subject fixates on a visual target. This embodiment of a method can include step 901, aligning an eye of a subject with an image capture device, as described herein. Step 902, capturing an image of at least one of the subject's eyes using the image capturing device. The image can include a landmark within at least one of the subject's eyes. Optionally, the landmark can be a feature within at least one of the subject's eyes such as a blood vessel, for example. This disclosure contemplates that landmarks other than blood vessels can be used such as a feature of the iris, the visible portion of the iris, the midpoint of the pupil, or the midpoint of the visible iris, and the like. The landmark can be any feature captured and identifiable within the captured image. The method can also include Step 904, analyzing the image to identify a position of the landmark within at least one of the subject's eyes, and Step 906, determining a phoria measurement based on the position of the landmark within at least one of the subject's eyes.

As used herein, at least one of the subject's eyes can be the subject's left eye or right eye. Optionally, the phoria measurement can be made based on the subject's left eye or right eye. Alternatively, at least one of the subject's eyes can be the subject's left eye and right eye. Optionally, the phoria measurement can be made based on the subject's left eye and right eye. This disclosure contemplates that the phoria measurement based on the subject's left eye and right eye can be the same or different.

As used herein, at least one of the subject's eyes can be the subject's left eye or right eye. Alternatively, at least one of the subject's eyes can be the subject's left eye and right eye. This disclosure contemplates that the optical qualities based on the subject's left eye and right eye can be the same or different.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method comprising:
aligning an eye with an image capture device;
capturing an image of the eye with the image capture device;
transmitting the captured image of the eye to a computing device;
detecting, using the computing device, light reflected out of an eye of a subject from a retina of the eye of the subject; and
making a determination about the eye of the subject based upon the reflected light.

2. The method of claim 1, wherein making a determination about the eye of the subject based upon the reflected light comprises making a determination based at least in part on an aspect of the reflected light.

3. The method of claim 1, wherein making a determination about the eye of the subject based upon the reflected light comprises making a determination based at least in part on a brightness and one or more colors of the reflected light.

4. The method of claim 1, wherein making a determination about the eye of the subject based upon the reflected light comprises making a determination of a refractive error for the eye of the subject based at least in part on an aspect of the reflected light.

5. The method of claim 1, wherein making a determination about the eye of the subject based upon the reflected light comprises making a determination of a refractive error for the eye of the subject based at least in part on a brightness and one or more colors of the reflected light.

6. The method of claim 1, wherein detecting, using the computing device, light reflected out of an eye of a subject from a retina of the eye of the subject further comprises:
capturing, using the image capture device, the image of the eye of a subject, wherein non-relevant reflections from the eye of the subject are managed while capturing the image;
determining, using the computing device, an overall intensity of light from a plurality of pixels located within the at least a portion of a pupil captured in the image;
determining, using the computing device, a first intensity of a first color from the plurality of pixels located within the at least a portion of a pupil of the eye of the subject captured in the image;
determining, using the computing device, a second intensity of a second color from the plurality of pixels located within the at least a portion of the pupil of the eye of the subject captured in the image; and
comparing, by the computing device, a relative intensity of the first color and a relative intensity of the second color, wherein said comparison and said overall intensity are used to make the determination about the eye of the subject based upon the reflected light.

7. The method of claim 1, wherein the image capture device comprises a smart phone or other mobile computing device having a camera.

8. The method of claim 7, wherein the eye is aligned with the image capture device using a software application executing on the smart phone or other mobile computing device having a camera.

9. The method of claim 8, wherein the software application executing on the smart phone or other mobile computing device having a camera provides visual and/or audible indicators through peripherals of the smart phone or other mobile computing device having a camera to align the eye with the image capture device.

10. A method comprising:
aligning an eye of a subject with an image capture device;
capturing, using the image capture device, an image of the eye of the subject, wherein non-relevant reflections from a cornea and a lens of the eye of the subject are managed while capturing the image;
pre-processing at least a portion of the image of the eye of the subject;
transmitting the pre-processed portion of the image of the eye of the subject to a computing device;
determining, using the computing device, an overall intensity of light from a plurality of pixels located within at least a portion of a pupil captured in the pre-processed portion of the image, wherein the plurality of pixels comprise red, green, and blue pixels;
determining, using the computing device, an average red intensity from the plurality of pixels located within the at least a portion of the pupil captured in the pre-processed portion of the image;
determining, using the computing device, an average blue intensity from the plurality of pixels located within the at least a portion of a pupil captured in the pre-processed portion of the image; and
determining, by the computing device, using the average red intensity, the average blue intensity and the determined overall intensity an optical quality of the eye.

11. The method of claim 10, wherein capturing, using the image capture device, the image of the eye of the subject comprises capturing a first image with the image capture device through a spectacle lens or a contact lens while the subject is wearing the spectacle lens or the contact lens over the eye and capturing a second image with the image capture device while the subject is not wearing the spectacle lens or the contact lens over the eye and the first image is compared to the second image and the determined optical quality of the eye is based on the comparison and comprises an estimated prescription for the spectacle lens or the contact lens.

12. The method of claim 10, wherein the method further comprises:
making a first determined optical quality about the eye of the subject based upon the reflected light from a first plurality of pixels located within the at least a portion of the pupil of the eye of the subject captured in the pre-processed portion of the image;
making a second determined optical quality about the eye from a second plurality of pixels located within the at least a portion of the pupil of the eye of the subject captured in the pre-processed portion of the image, wherein the second plurality of pixels are a subset of the first plurality of pixels;
making a third determined optical quality about the eye from a third plurality of pixels located within the at least a portion of the pupil of the eye of the subject captured in the pre- processed portion of the image, wherein the third plurality of pixels are a subset of the first plurality of pixels and are separate from the second plurality of pixels; and
comparing the first determined optical quality, the second determined optical quality and the third determined optical quality to make the determined optical quality about the eye of the subject based upon the reflected light.

13. The method of claim 10, wherein the image capture device comprises a smart phone or other mobile computing device having a camera.

14. The method of claim 13, wherein the eye is aligned with the image capture device using a software application executing on the smart phone or other mobile computing device having a camera.

15. The method of claim 14, wherein the software application executing on the smart phone or other mobile computing device having a camera provides visual and/or audible indicators through peripherals of the smart phone or other mobile computing device having a camera to align the eye with the image capture device.

16. A system comprised of:
an apparatus, said apparatus comprised of:
an image capture device;
a memory;
a network interface; and
a processor in communication with the memory, the image capture device, and the network interface, wherein the processor executes computer-readable instructions stored in the memory that cause the processor to:
assist in aligning an eye of a subject with the image capture device;
capture, using the image capture device, an image of an eye of a subject, wherein non-relevant reflections from the eye of the subject are managed while capturing the image;
pre-process the captured image of the eye of the subject; and
transmit, using the network interface, the pre-processed captured image of the eye of the subject to a computing device, wherein a processor of the computing device executes computer-readable instructions stored in a memory of the computing device that cause the processor of the computing device to:
detect, from the pre-processed image of the eye of the subject, light reflected out of the eye of a subject from a retina of the eye of the subject; and
make a determination about the eye of the subject based upon the detected reflected light.

17. The system of claim 16, wherein the processor of the computing device executing computer-readable instructions stored in the memory of the computing device that cause the processor of the computing device to detect light reflected out of an eye of a subject from a retina of the eye of the subject further comprises the processor of the computing device executing computer-readable instructions stored in the memory of the computing device that cause the processor of the computing device to:
determine an overall intensity of light from a plurality of pixels located within the at least a portion of a pupil captured in the pre-processed image;
determine a first intensity of a first color from the plurality of pixels located within the at least a portion of a pupil of the eye of the subject captured in the pre-processed image;
determine a second intensity of a second color from the plurality of pixels located within the at least a portion of the pupil of the eye of the subject captured in the pre-processed image; and
determine using a relative intensity of the first color and a relative intensity of the second color and the overall intensity the determination about the eye of the subject based upon the reflected light.

18. The system of claim 16, wherein the image capture device comprises a smart phone or other mobile computing device having a camera.

19. The system of claim 18, wherein the eye is aligned with the image capture device using a software application executing on the smart phone or other mobile computing device having a camera.

20. The system of claim 19, wherein the software application executing on the smart phone or other mobile computing device having a camera provides visual and/or audible indicators through peripherals of the smart phone or other mobile computing device having a camera to align the eye with the image capture device.
